(19) United States
(12) Patent Application Publication
(10) Pub. No.: US 2014/0058790 A1
(43) Pub. Date: Feb. 27, 2014

(54) DEUTERATED PEPTIDES

(71) Applicants: Michael H.B. Stowell, Boulder, CO (US); Mikhail Plam, Boulder, CO (US)

(72) Inventors: Michael H.B. Stowell, Boulder, CO (US); Mikhail Plam, Boulder, CO (US)

(73) Assignee: AmideBio LLC, Boulder, CO (US)

(21) Appl. No.: 13/989,336

(22) PCT Filed: Nov. 30, 2011
(86) PCT No.: PCT/US11/62733

§ 371 Date: Nov. 12, 2013

Related U.S. Application Data
(60) Provisional application No. 61/418,774, filed on Dec. 1, 2010.

(51) Int. Cl. C12P 21/00; G06Q 30/02; C12N 15/63

(52) U.S. Cl. CPC C12P 21/00; C12N 15/63; G06Q 30/0201
USPC 705/7.29; 435/69.7; 435/69.4; 435/69.6; 435/69.5; 435/69.51; 435/69.2; 435/226; 435/209; 435/183; 530/300; 530/383; 530/397; 530/399; 530/351; 530/381; 435/252.33; 435/254.2; 435/254.21; 435/254.23; 435/325; 435/348; 435/358; 435/357; 435/365; 435/367

(57) ABSTRACT

Methods and compositions described herein relate to processes for the production of deuterated peptides, and the deuterated peptides produced accordingly. Deuterated peptides produced according to methods and compositions described herein may be produced more efficiently than such peptides produced according to prior art processes. The production process of according to methods and compositions described herein may lead to advantages in yield, purity, and/or price for deuterated peptides. Methods of marketing deuterated peptides are also disclosed.

This application claims priority from U.S. Provisional Application 61/418,774, filed Dec. 1, 2010, the entire disclosure of which is incorporated by reference in its entirety.

### FIELD OF THE INVENTION

Methods and compositions described herein relate to processes for the production of deuterated peptides, and the deuterated peptides produced accordingly. Deuterated peptides produced according to methods and compositions described herein may be produced more efficiently than such peptides produced according to prior art processes. The production process according to methods and compositions described herein may lead to advantages in yield, purity, and/or price for deuterated peptides. Methods of marketing deuterated peptides are also disclosed.

### BACKGROUND OF THE INVENTION

Peptide market values are generally grouped within five broad categories in the life sciences field: cytokines, enzymes, hormones, monoclonal antibodies, and vaccines. Each of these categories is undergoing high growth rates. Moreover, peptide markets are likely to continue to grow as additional opportunities are developed for peptides as therapeutics, reagents in basic research, and diagnostic platforms.

Peptides are becoming increasingly useful in basic research and clinical practice for various reasons. Interest in peptides can be attributed in part to their role as mediators in many biological pathways and to their unique intrinsic properties. For example, many peptides have high specificity for their target with low non-specific binding to molecules that are not targeted, thus minimizing drug-drug interactions, and many peptides show low accumulation in tissues over time, thus reducing side effects. Moreover, peptides are often broken down in vivo to their constituent amino acids, thus reducing the risk of complications due to toxic metabolic intermediates.

While advances in the field of peptide science have led to impressive commercial growth, several barriers remain to be overcome. For example, peptides tend to have delivery and stability problems compared to traditional small molecule therapeutics. In addition, one major barrier to increased use of peptides is the cost of the peptides themselves, which is generally significantly higher than the cost of producing small molecule therapeutics. High prices are an even bigger barrier to obtaining peptides when the peptide is used for research purposes.

Due to the importance of peptides, their high price, and their stability problems, there is a persistent and long-felt yet unfilled need for producing peptides with improved properties at lower cost. The industrial and medical use of peptides has created a need for an improved means of production and purification, where the improvement may be in efficiency, price, and/or properties of the peptide product. Accordingly, methods and compositions described herein are directed to the production of deuterated peptides, and the deuterated peptides produced. Methods of marketing deuterated peptides are also disclosed.

### SUMMARY OF THE INVENTION

In various embodiments, methods and compositions described herein are directed to producing a deuterated target peptide. In one embodiment, a fusion peptide is produced comprising an affinity tag, a cleavable tag, and the target peptide, followed by binding of the fusion peptide to an affinity material, cleaving the fusion peptide to release the target peptide; and removing the target peptide from the affinity material. In another embodiment, the fusion peptide is deuterated. In general, following binding of the fusion peptide to an affinity material, the affinity material is washed to remove unbound material. Moreover, following removal of the target peptide from the affinity material, the target peptide may be further modified or packaged for distribution.

In various embodiments, a peptide is selected from the group consisting of amyloid beta, calcitonin, enfuvirtide, epoetin, epoetin delta, erythropoietin, exenatide, factor VIII, factor X, glucocerebrosidase, glucagon-like peptide-1 (GLP-1), granulocyte-colony stimulating factor (G-CSF), human growth hormone (hGH), insulin, insulin A, insulin B, insulin-like growth factor 1 (IGF-1), interferon, liraglutide, somatostatin, teriparatide, and tissue plasminogen activator (TPA). In various embodiments, the peptide is selected from amyloid beta, enfuvirtide, exenatide, insulin, and teriparatide.

The fusion peptide may be produced in a variety of methods. In one embodiment, the fusion peptide is produced in a bacterial expression system, such as an E. coli expression system. In alternate embodiments, the expression system is a yeast expression system, an insect cell expression system, or a mammalian expression system.

In various embodiments, the deuterated fusion peptide according to methods and compositions described herein further comprises an inclusion-body directing peptide. In such embodiments, prior to the binding of the fusion peptide to the affinity material, the fusion peptide may be isolated from the expression system by separation of inclusion bodies from the remainder of the cell in the expression system. In another embodiment, an inclusion body comprises deuterated peptide. Following initial isolation, the fusion peptide may be solubilized to allow further handling. In various embodiments, the inclusion-body directing peptide is selected from the group consisting of inclusion-body directing peptide is a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, or an inclusion-body directing functional homolog of BRCA2.

In various embodiments, the affinity tag is selected from the group consisting of poly-histidine, poly-lysine, poly-aspartic acid, or poly-glutamic acid. Moreover, the cleavable tag may be selected from the group consisting of Trp, His-Met, Pro-Met, and an unnatural amino acid. In the event of more than one cleavable tag in the fusion peptide, the various cleavable tags may be orthogonal, i.e. have different reactivity with any particular cleavage agent. In various embodiments, the cleaving step is performed with an agent selected from the group consisting of NBS, NCS, or Pd(H₂O)₄.

Methods and compositions described herein are also directed to evaluating the commercial market for a target peptide comprising a) producing a target peptide according to the methods described herein; b) making sample amounts of the target peptide available for no cost or minimal cost; and c) measuring the number of requests for the target peptide over a period of time.

Methods and compositions described herein are directed to peptide of greater than or equal to about 99% purity. Also, methods and compositions described herein are directed to vectors for use in expression systems for the production of target peptides. For example, vectors disclosed herein may include a nucleotide sequence encoding an affinity tag; a nucleotide sequence encoding a cleavable tag; and a nucleotide sequence encoding a target peptide; wherein the nucleotides are arranged in operable combination and further wherein expression of the operable combination results in a fusion protein comprising an affinity tag, a cleavable tag, and a target peptide. Additional embodiments are directed to a cell comprising the vectors described herein as well as a fusion protein produced according to the methods described herein.

### BRIEF DESCRIPTION OF THE DRAWINGS

Various features of methods and compositions described herein are set forth with particularity in the appended claims. A better understanding of the features and advantages of methods and compositions described herein will be obtained by reference to the following detailed description that sets forth illustrative embodiments, in which the principles of methods and compositions described herein are utilized, and the accompanying drawings of which:

FIG. 1 diagrams a modified form of the commercially available pET-19b vector (pET-19bmhb1). Such vectors can be used to produce a KSI sequence flanked by two NcoI restriction sites and a histidine tag-tryptophan-β-amyloid (1-42) sequence flanked by two XhoI restriction sites.

FIG. 2 illustrates activation of transcription in a commercially available pBAD promoter via the addition of L-arabinose. Arabinose binds to AraC (“C” in the diagram) and causes the protein to release the O₂ site and bind the I₂ site which is adjacent to the I₁ site. This releases the DNA loop and allows transcription to begin. A second level of control is present in the cAMP activator protein (CAP)-cAMP complex, which binds to the DNA and stimulates binding of AraC to I₁ and I₂. Basal expression levels can be repressed by introducing glucose to the growth medium, which lowers cAMP levels and in turn decreases the binding of CAP, thus decreasing transcriptional activation.

FIG. 3A-D presents four embodiments of amino acid sequences for ketosteroid isomerase.

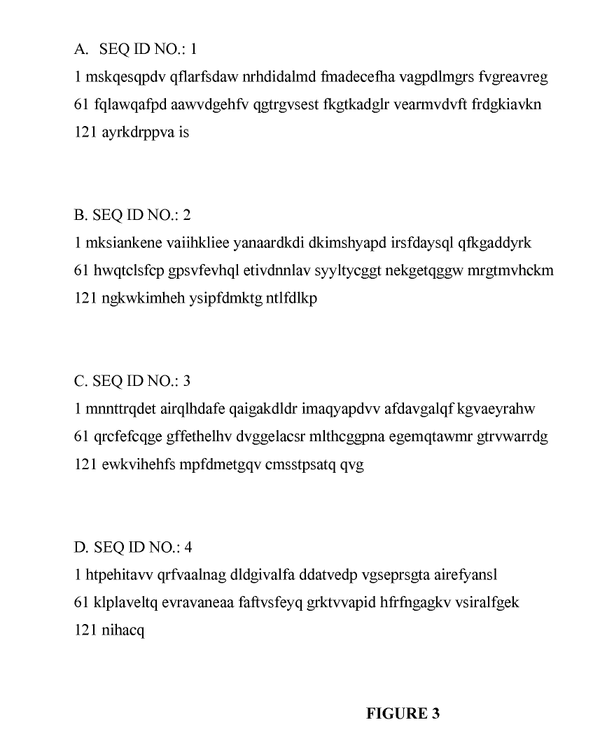

FIG. 4 presents one embodiment of a nucleic acid sequence for ketosteroid isomerase.

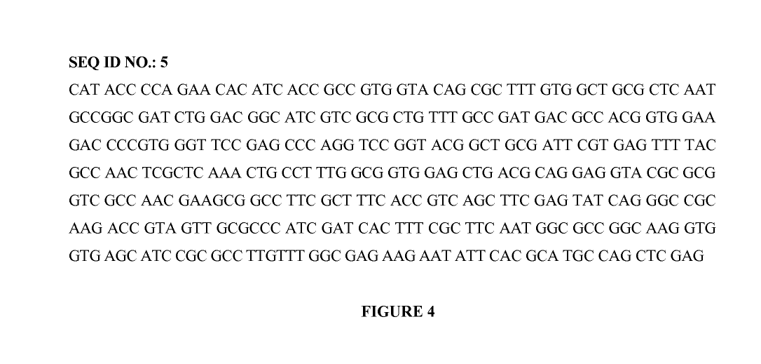

FIG. 5 illustrates one embodiment of an immobilized Ni-NTA resin binding to a 6×His tag on a protein.

FIG. 6 illustrates one possible mechanism for the selective cleavage of tryptophan peptide bonds with NBS (N-bromosuccinimide). According to the mechanism, the active bromide ion halogenates the indole ring of the tryptophan residue followed by a spontaneous dehalogenation through a series of hydrolysis reactions. These reactions lead to the formation of an oxindole derivative which promotes the cleavage reaction. In FIG. 6, Z-Trp-Y is cleaved at the carboxy terminus of the Trp residue to yield a modified Z-Trp and a free amino group on Y (i.e., H₂N—Y).

FIG. 7 presents the chemical structures of a variety of unnatural amino acids that have been incorporated into peptides and proteins by cell systems through genetic modification of the cell systems. See Wang, et al., (2009) Chem. Biol. 16(3):323-36.

### DETAILED DESCRIPTION OF THE INVENTION

Described herein are methods and compositions comprising a peptide containing one or more isotopes. In one embodiment, the isotope is deuterium, which is an isotope of hydrogen with a nucleus comprising one neutron and one proton. In another embodiment, one or more deuterated peptides may be produced by ribosomal synthesis methods described herein. In another embodiment, one or more deuterated peptides may be produced by solid peptide synthesis methods described herein. In another embodiment, one or more deuterated peptides may be produced by non-ribosomal synthesis methods described herein.

Without wishing to be bound by theory, it is believed that replacement of at least one hydrogen with a deuterium isotope will provide peptides with different properties. For example, deuterated peptides according to the methods and compositions described herein may have improved pharmacokinetic properties without significantly altering the biological activity of the peptides. Variously, deuterated peptides as described herein may be useful for diagnostic purposes or analytic purposes. Where small molecules labeled with deuterium or otherwise with isotopes such as ¹³C, ¹⁴C, ¹⁵N, ³¹P have been used in diagnostic studies to trace metabolic pathways or degradation pathways of a drug, deuterated peptides as described herein may also be used in a corresponding fashion.

Disclosed herein provide methods for producing fusion peptides that can be purified and cleaved into desired peptides, and the peptides produced according to the methods. In various embodiments, the method includes induction, deuteration, inclusion body isolation, affinity column purification, and chemical cleavage. In various embodiments, methods and compositions described herein utilize an expression vector to make the peptides described herein. In some aspects, by combining molecular expression technologies that employ genetically-malleable microorganisms such as E. coli cells to synthesize a peptide of interest with post-expression isolation and modification, one can deuterate and synthesize a desired peptide rapidly and efficiently. In various embodiments, methods and compositions described herein produce deuterated fusion peptides that can be purified using affinity separation and cleaved with a chemical reagent to release a target peptide.

In various embodiments, methods and compositions described herein are directed to a vector that encodes an inclusion body targeting sequence, an affinity tag to facilitate purification, and a specific amino acid sequence that facilitates selective chemical cleavage. Variously, the inclusion body targeting amino acid sequence comprises from about 1 to about 125 amino acids of a ketosteroid isomerase protein. The affinity tag sequence may comprise a poly-histidine, a poly-lysine, poly-aspartic acid, or poly-glutamic acid. In one embodiment, the vector further comprises an expression promoter located on the 5′ end of the affinity tag sequence. In one embodiment, methods and compositions described herein are directed to a vector that codes for a specific sequence that facilitates selective chemical cleavage to yield a peptide of interest following purification. Such chemically cleavable amino acid sequences include Trp, His-Met, or Pro-Met.

In one embodiment, methods and compositions described herein utilize a peptide expression vector, comprising: a) a first nucleotide sequence encoding an affinity tag amino acid sequence; b) a second nucleotide sequence encoding an inclusion body targeting amino acid sequence; c) a third nucleotide sequence encoding a chemically cleavable amino acid sequence; and d) a promoter in operable combination with the first, second, and third nucleotide sequences.

In one embodiment, methods and compositions described herein produce a deuterated peptide of commercial or therapeutic interest comprising the steps of: a) cleaving a vector with a restriction endonuclease to produce a cleaved vector; b) ligating the cleavage site to one or more nucleic acids, wherein the nucleic acids encode a desired peptide having at least a base overhang at each end configured and arranged for ligation with the cleaved vector to produce a second vector suitable for expression of a fusion peptide; c) transforming the second vector into suitable host cell; d) incubating the host cell under conditions suitable for expression of deuterated fusion peptide; e) isolation of inclusion bodies from the host cell; f) solubilization and extraction of the fusion peptide from the inclusion bodies; g) binding of the fusion peptide to a suitable affinity material; h) washing of bound fusion peptide to remove impurities; and i) cleaving the fusion peptide to release the said target peptide.

Peptides produced by methods and compositions described herein may have significantly lower costs and/or other advantageous features. These potentially cheaper costs may lie not only in less expensive raw materials required for production, but also may lie in less chemical waste which is generated compared to the traditional process of solid phase peptide synthesis, or in more efficient processing to achieve a certain purity, thus lowering the cost of the material. Furthermore, the exclusion of a waste stream may be particularly beneficial to the environment. In various embodiments, processes according to methods and compositions described herein provide a high yield of deuterated peptide with high purity. In various embodiments, deuterated peptides produced according to methods and compositions described herein may be R&D grade peptides or clinical grade therapeutics.

### DEFINITIONS

Unless defined otherwise, all technical and scientific terms used herein have the same meaning as commonly understood by a person of ordinary skill in the art.

As used herein, the term “peptide” is intended to mean any polymer comprising amino acids linked by peptide bonds. The term “peptide” is intended to include polymers that are assembled using a ribosome as well as polymers that are assembled by enzymes (i.e., non-ribosomal peptides) and polymers that are assembled synthetically. In various embodiments, the term “peptide” may be considered synonymous with “protein,” or “polypeptide.” In various embodiments, the term “peptide” may be limited to a polymer of greater than 50 amino acids, or alternatively, 50 or fewer amino acids. In various embodiments, the term “peptide” is intended to include only amino acids as monomeric units for the polymer, while in various embodiments, the term “peptide” includes additional components and/or modifications to the amino acid backbone. For example, in various embodiments, the term “peptide” may be applied to a core polymer of amino acids as well as derivatives of the core polymer, such as core polymers with pendant polyethylene glycol groups or core polymers with amide groups at the amino or carboxy terminus of the amino acid chain.

As used herein, “consisting essentially of” may exclude those features not listed herein that would otherwise alter the operation of methods and compositions described herein. However, the use of the phrase “consisting essentially of” does not exclude features that do not alter the operation of the required components.

The term “polymer” is a molecule (or macromolecule) composed of repeating structural units connected by covalent chemical bonds.

A “patient,” “subject” or “host” to be treated with methods and compositions described herein may mean either a human or non-human animal. The term “mammal” is known in the art, and exemplary mammals include human, primate, bovine, porcine, canine, feline, and rodent (e.g., mice and rats).

### I. Target Peptides

Methods and compositions described herein are applicable to a wide range of deuterated peptides as the isolated product, which may be referred to as target peptides. Peptides produced according to methods and compositions described herein may be homologous to naturally-occurring peptides, non-naturally-occurring peptides, or naturally-occurring peptides with non-natural substitutions, deletions, or additions. In various embodiments, the target peptide may be modified chemically or biologically following isolation to yield a derivative of the target peptide, such as a target peptide with one or more carboxamide groups in place of free carboxy groups. Non-natural peptide may also include, but is not limited to, peptide comprising one or more man-made modifications such as modified amino acid, biotin, phosphorylation, fluorescein, glycosylation and the like.

In various embodiments, the peptide is selected from vaccines, antibodies, recombinant hormones and proteins, interferons, interleukins, and growth factors. In some embodiments, the target peptide is fifty or fewer amino acids. In some embodiments, the target peptide is greater than fifty amino acids.

Methods and compositions described herein are applicable to a variety of peptides. As methods and compositions described herein take advantage of properties inherently associated with peptides, without being bound by theory, methods and compositions described herein may produce a deuterated form of virtually any peptide found either in nature or not. Further non-limiting embodiments include peptides and analogs thereof selected from the group consisting of angiotensin, arginine vasopressin (AVP), AGG01, amylin (IAPP), amyloid beta, N-acetylgalactosamine-4-sulfatase (rhASB; galsulfase), avian pancreatic polypeptide (APP), B-type natriuretic peptide (BNP), calcitonin peptides, calcitonin, colistin (polymyxin E), colistin copolymer 1 (Cop-1), cyclosporin, darbepoetin, PDpoetin, dornase alfa, eledoisin, β-endorphin, enfuvirtide, enkephalin pentapeptides, epoetin, epoetin delta, erythropoietin, exenatide, factor VIII, factor X, follicle-stimulating hormone (FSH), alpha-galactosidase A (Fabrazyme), Growth Hormone Releasing Hormone 1-24 (GHRH 1-24), β-globin, glucagon, glucocerebrosidase, glucagon-like peptide-1 (GLP-1), granulocyte-colony stimulating factor (G-CSF), granulocyte macrophage-colony stimulating factor (GM-CSF), growth hormone, Hepatitis B viral envelope protein, human growth hormone (hGH), insulin, insulin A, insulin B, insulin-like growth factor 1 (IGF-1), interferon, kassinin, alpha-L-iduronidase (rhIDU; laronidase), lactotripeptides, leptin, liraglutide (NN2211, VICTOZA), luteinizing-hormone-releasing hormone, methoxy polyethylene glycol-epoetin beta (MIRCERA), myoglobin, neurokinin A, neurokinin B, NN9924, NPY (NeuroPeptide Y), octreotide, pituitary adenylate cyclase activating peptide (PACAP), parathyroid hormone (PTH), Peptide Histidine Isoleucine 27 (PHI 27), proopiomelanocortin (POMC) peptides, prodynorphin peptides, polymyxins, polymyxin B, Pancreatic Polypeptide (PPY), Peptide YY (PYY), secretin, somatostatin, Substance P, teriparatide (FORTEO), tissue plasminogen activator (TPA), thrombospondins (TSP), ubiquitin, urogastrone, Vasoactive Intestinal Peptide (VIP, or PHM27), and viral envelope proteins. In various embodiments, the target peptide is selected from amyloid beta, calcitonin, enfuvirtide, epoetin, epoetin delta, erythropoietin, exenatide, factor VIII, factor X, glucocerebrosidase, glucagon-like peptide-1 (GLP-1), granulocyte-colony stimulating factor (G-CSF), human growth hormone (hGH), insulin, insulin A, insulin B, insulin-like growth factor 1 (IGF-1), interferon, liraglutide, somatostatin, teriparatide, and tissue plasminogen activator (TPA). In various embodiments, the target peptide is selected from amyloid beta and insulin.

In various embodiments, a deuterated peptide may represent a portion of a protein described herein or the whole protein. In various embodiments, a deuterated peptide may have a sequence homologous to a portion of a protein or the whole protein. For example, a deuterated peptide may be about 95% homologous to a portion of human insulin in comparison of amino acid sequence. The percentage of sequence homology between a deuterated peptide and a naturally occurring wild type counterpart may be about 50%, 55%, 60%, 65%, 70%, 75%, 80%, 85%, or 90%. In another embodiment, a deuterated peptide may be identical to a naturally occurring wild type counterpart in amino acid sequence but may not be identical in other aspects such as glycosylation or other post-translational modifications.

In various embodiments, the target peptide is a hormone. For example, in various embodiments, the target peptide is selected from the group consisting of Activin, inhibin, Adiponectin, Adipose derived hormones, Adrenocorticotropic hormone, Afamelanotide, Agouti signalling peptide, Allatostatin, Amylin, Angiotensin, Atrial natriuretic peptide, Bovine somatotropin, Bradykinin, Brain-derived neurotrophic factor, CJC-1295, Calcitonin, Ciliary neurotrophic factor, Corticotropin-releasing hormone, Cosyntropin, Endothelin, Enteroglucagon, Follicle-stimulating hormone, Gastrin, Gastroinhibitory peptide, Glucagon, Glucagon hormone family, Glucagon-like peptide-1, Gonadotropin, Granulocyte colony-stimulating factor, Growth hormone, Growth hormone releasing hormone, Hepcidin, Human chorionic gonadotropin, Human placental lactogen, Incretin, Insulin, Insulin glargine, Insulin lispro, Insulin aspart, Insulin-like growth factor 2, Insulin-like growth factor, Leptin, Liraglutide, Luteinizing hormone, Melanocortin, Melanocyte-stimulating hormone, Melanotan II, Minigastrin, N-terminal prohormone of brain natriuretic peptide, Nerve growth factor, Neurotrophin-3, NPH insulin, Obestatin, Orexin, Osteocalcin, Pancreatic hormone, Parathyroid hormone, Peptide YY, Peptide hormone, Plasma renin activity, Pramlintide, Preprohormone, Proislet Amyloid Polypeptide, Prolactin, Relaxin, Renin, Salcatonin, Secretin, Sincalide, Teleost leptins, Thyroid-stimulating hormone, Thyrotropin-releasing hormone, Urocortin, Urocortin II, Urocortin III, Vasoactive intestinal peptide, and Vitellogenin.

In various embodiments, a non-deuterated form of target peptide is already commercially available through a production process that differs from methods and compositions described herein. While not wishing to be bound by theory, it is believed that peptides produced according to methods and compositions described herein will have differing levels of residual components from the process of production. For example, in comparison with peptides of the same sequence produced according to conventional recombinant processes, peptides produced according to methods and compositions described herein may be expected to have fewer residual cellular contaminants upon initial purification. Alternatively, in comparison with peptides of the same sequence produced by conventional synthetic processes, peptides produced according to methods and compositions described herein may be expected to have fewer residual chemical contaminants upon initial purification. Various commercially available peptides may be found in paper catalogs or in online catalogs, for example, from Sigma-Aldrich (<<sigmaaldrich.com/life-science/cell-biology/peptides-and-proteins.html>>), California Peptide (<<californiapeptide.com/peptide_catalog_table>>), CPC Scientific (<<cpcscientific.com/products/browseCatalog.asp>>), and Bachem (<<shop.bachem.com/ep6sf/index.ep>>), the contents of each of which are hereby incorporated by reference.

In various embodiments, target peptides do not include tryptophan in their sequence.

In one aspect, a peptide disclosed herein refers to a peptide containing one or more isotope. In one embodiment, an isotope is deuterium. In another embodiment, a deuterium forms a covalent molecular bond with a carbon atom of an amino acid. In another embodiment, a deuterium forms a molecular bond with a nitrogen atom of an amino acid. In another embodiment, one or more hydrogen atoms of an amino acid are substituted with deuterium. In another embodiment, the substitution occurs in a particular hydrogen atom. In another embodiment, the substitution is random. In one embodiment, the deuterium is non-exchangable, or the deuterium does not dissociate from the atom to which is connected in an aqueous solution.

In one aspect, a peptide described herein is deuterated by incorporating various numbers of deuterated amino acids. The number of deuterated amino acid in a peptide may be one, two or more. In various embodiments, the percentage of deuterated amino acid in a peptide may be as little as 1 amino acid in the peptide, or in various embodiments, may be 1% of the total number of amino acids comprising a peptide. In another embodiment, every amino acid comprising a peptide may be deuterated. In another embodiment, a particular kind of amino acid is deuterated in a peptide. For example, if a peptide comprises five Glycine residues, all Glycine residues in the peptide may be deuterated. In another embodiment, the most N-terminally located amino acid is deuterated. In another embodiment, the most C-terminally located amino acid of is deuterated. In another embodiment, side-chains are deuterated but not the back-bone of a peptide. In another embodiment, the hydrogen atoms attached to the C—N—O back-bone are deuterated but not the hydrogen atoms in the side-chain. In another embodiment, an amino acid with bulky side chain is deuterated. Examples of amino acids having bulky side chains include, but are not limited to, isoleucine, tryptophan, phenylalanine, tyrosine, methionine, aspartic acid, asparagine, glutamic acid, glutamine, proline, histidine, arginine, and lysine. In another embodiment, an amino acid with small side chain, such as alanine, leucine, or glycine, is deuterated. In various embodiments, a percentage of one or more amino acids is deuterated. For example, where the deuterium-labeled amino acid is glycine, a percentage of the glycines ranging from 1% to 100% of the glycines in the peptide may be labeled, including more than 10%, more than 20%, more than 30%, more than 40%, more than 50%, more than 60%, more than 70%, more than 80%, and more than 90% of the glycines with deuterium labels. Alternatively, less than 10%, less than 20%, less than 30%, less than 40%, less than 50%, less than 60%, less than 70%, less than 80%, and less than 90% of the glycines may be labeled with deuterium labels. In various embodiments, the exemplary glycine as described above may be replaced with any other amino acid or amino acid analog.

In one aspect, a deuterated peptide has nearly identical physiochemical properties as non-deuterated peptide but behaves differently in vivo. In one embodiment, a deuterated peptide has longer in vivo degradation time than its non-deuterated counterpart. In another embodiment, one or more carbon-deuterium bonds (C-D bonds) of a deuterated peptide are located within an active site for an enzyme capable of cleaving the peptide. In another embodiment, the C-D bonds slow the rate of enzymatic cleavage. In another embodiment, one or more carbon-deuterium bonds (C-D bonds) of a deuterated peptide at or near a protein-protein interaction interface between the peptide and a protein. In another embodiment, a protein-protein interaction is a ligand-protein interaction or a receptor-ligand interaction. In one embodiment, the deuterium label is located at a site of non-specific binding, for example, a site of non-specific binding with albumin.

Described herein is a ligand comprising a deuterated peptide. In one embodiment, a deuterated ligand interacts with a protein. An example of the protein includes, but is not limited to, a molecule involved in a metabolic pathway such as an enzyme, a cell surface molecule such as a receptor or a channel, a cytosolic or nuclear protein involved in a cell signaling pathway, a cytosolic or nuclear protein involved in DNA metabolism, and other proteins involved in cellular activities such as degradation, exocytosis, endocytosis, apoptosis, cell division, and the like. In another embodiment, a deuterated ligand attenuates ligand-protein interactions described herein. In another embodiment, a deuterated ligand prolongs ligand-protein interactions described herein. In another embodiment, a deuterated ligand induces ligand-protein interactions described herein. In another embodiment, a deuterated ligand inhibits ligand-protein interactions described herein. Described herein is a dosage form comprising one or more deuterated peptides. In one embodiment, a dosage form comprising one or more deuterated peptides may be used for clinical purpose. A clinical purpose includes, but is not limited to, diagnosis, prognosis, therapy, clinical trial, and clinical research. In one embodiment, a deuterated peptide is used for studying pharmacokinetics/pharmacodynamics.

In another embodiment, a dosage form may be formulated for a particular delivery route. A delivery route includes, but is not limited to, oral, nasal, rectal, intravascular, intraperitoneal, subcutaneous, ocular, dermal and the like. A dosage form may be packaged as tablet, gel, aerosol, fluid, particulate, capsule, powder, film, or a coating. A dosage form may also be delivered via a stent or other invasive device such as an implant. In another embodiment, a deuterated peptide is lyophilized. In another embodiment, a deuterated peptide is in solution. In another embodiment, a deuterated peptide is provided as a concentrate accompanied with an appropriate dilution solution and instruction. In another embodiment, a deuterated peptide is in powdered form. In another embodiment, a deuterated peptide is provided as gel or in other viscous material such as polyethylene glycol. In another embodiment, a deuterated peptide is provided in a micelle such as a liposome.

In one embodiment, a dosage form comprises a mixture of deuterated and non-deuterated peptide. In another embodiment, a dosage form comprises a formulation having two physically separated compartments wherein a deuterated form occupies one compartment and non-deuterated form occupies another compartment. The ratio of deuterated form to non-deuterated form may be about 1:2, 1:3, 1:4, 1:5, 1:7, 1:9, 1:10, 1:15, 1:20, 1:30, 1:50, 1:70, 1:100, 1:500, 1:1000 or vice versa. A deuterated form may comprise 1%, 2%, 4%, 8%, 9.5%, 11.8%, 14.1%, 16.4%, 18.7%, 21%, 23.3%, 25.6%, 27.9%, 30.2%, 32.5%, 34.8%, 37.1%, 39.4%, 41.7%, 44%, 46.3%, 48.6%, 50.9%, 53.2%, 55.5%, 57.8%, 60.1%, 62.4%, 64.7%, 67%, 69.3%, 71.6%, 73.9%, 76.2%, 78.5%, 80.8%, 83.1%, 85.4%, 87.7%, 90%, 92.3%, 94.6%, 96.9%, 99.2% of the total amount of peptide in a dosage form. In another embodiment, a deuterated form and non-deuterated form are released to an animal upon dissolution with varying pharmacokinetic properties. For example, a dosage form may provide immediate release of a deuterated form and slow, sustained release of a non-deuterated form or vice versa.

### II. Inclusion-Body Directing Peptides

Inclusion bodies are composed of insoluble and denatured forms of a peptide and are about 0.5-1.3 μm in diameter. These dense and porous aggregates help to simplify recombinant protein production since they have a high homogeneity of the expressed protein or peptide, result in lower degradation of the expressed protein or peptide because of a higher resistance to proteolytic attack by cellular proteases, and are easy to isolate from the rest of the cell due to differences in their density and size relative to the other cellular components. In various embodiments, the presence of inclusion bodies permits production of increased concentrations of the expressed protein or peptide due to reduced toxicity by the protein or peptide upon segregation into an inclusion body. Once isolated, the inclusion bodies may be solubilized to allow for further manipulation and/or purification.

An inclusion-body directing peptide is an amino acid sequence that helps to direct a newly translated protein or peptide into insoluble aggregates within the host cell. Prior to final isolation, in various embodiments, the target peptide is produced as a fusion peptide where the fusion peptide includes as part of its sequence of amino acids an inclusion-body directing peptide. Methods and compositions described herein are applicable to a wide range of inclusion-body directing peptides as components of the expressed fusion protein or peptide.

In various embodiments, the inclusion-body directing peptide is a keto-steroid isomerase (KSI) sequence, a functional fragment thereof, or a functional homolog thereof

In various embodiments, the inclusion-body directing peptide is a BRCA-2 sequence, a functional fragment thereof, or a functional homolog thereof.

In various embodiments, the inclusion-body directing peptide is a deuterated form of a keto-steroid isomerase (KSI) sequence, a functional fragment thereof, or a functional homolog thereof

In various embodiments, the inclusion-body directing peptide is a deuterated form of a BRCA-2 sequence, a functional fragment thereof, or a functional homolog thereof

### III. Affinity-Tag Peptides

According to methods and compositions described herein, a wide variety of affinity tags may be used. Affinity tags useful according to methods and compositions described herein may be specific for cations, anions, metals, or any other material suitable for an affinity column. In one embodiment, any peptide not possessing an affinity tag will elute through the affinity column leaving the desired fusion peptide bound to the affinity column via the affinity tag.

Specific affinity tags according to methods and compositions described herein may include poly-lysine, poly-histidine, poly-glutamic acid, or poly-arginine peptides. For example, the affinity tags may be 5-10 lysines, 5-10 histidines, 5-10 glutamic acids, or 5-10 arginines. In various embodiments, the affinity tag is a hexa-histidine sequence, hexa-lysine sequence, hexa-glutamic acid sequence, or hexa-arginine sequence. Alternatively, the HAT-tag (Clontech) may be used. In various embodiments, the affinity tag is a His-Trp Ni-affinity tag. Other tags known in the art may also be used. Examples of tags include, but are not limited to, Isopeptag, BCCP-tag, Myc-tag, Calmodulin-tag, FLAG-tag, HA-tag, MBP-tag, Nus-tag, GST-tag, GFP-tag, Thioredoxin-tag, S-tag, Softag, Streptavidin-tag, V5-tag, CBP-tag, and SBP-tag.

Without wishing to be bound by theory, it is believed that the histidine residues of a poly-histidine tag bind with high affinity to Ni-NTA or TALON resins. Both of these resins contain a divalent cation (Ni-NTA resins contain Mg²⁺; TALON resins contain Co²⁺) that forms a high affinity coordination with the His tag.

In various embodiments, the affinity tag has a pI (isoelectric point) that is at least one pH unit separate from the pI of the target peptide. Such difference may be either above or below the pI of the target peptide. For example, in various embodiments, the target peptide has a high pI, and the affinity tag has a pI that is at least one pH unit lower, at least two pH units lower, at least three pH units lower, at least four pH units lower, at least five pH units lower, at least six pH units lower, or at least seven pH units lower. Alternatively, the target peptide has a low pI, and the affinity tag has a pI that is at least one pH unit higher, at least two pH units higher, at least three pH units higher, at least four pH units higher, at least five pH units higher, at least six pH units higher, or at least seven pH units higher. In one embodiment, the target peptide has a pI of about 10 and the affinity tag has a pI of about 6.

In various embodiments, the affinity tag is contained within the native sequence of the inclusion body directing peptide. Alternatively, the inclusion body directing peptide is modified to include an affinity tag. For example, in one embodiment, the affinity tag is a KSI or BRCA2 sequence modified to include extra histidines, extra lysines, extra arginines, or extra glutamic acids.

In various embodiments, epitopes may be used such as FLAG (Eastman Kodak) or myc (Invitrogen) in conjunction with their antibody pairs.

### IV. Cleavable Tags

Methods and compositions described herein are applicable to a wide range of cleavable tags.

In various embodiments, the cleavable tag is a tryptophan at the amino terminus of the target peptide. Upon cleavage with a cleaving agent, the amide bond connecting the tryptophan to the target peptide is cleaved, and the target peptide is released from the affinity column.

In various embodiments, the cleavable tag is a tryptophan at the amino terminus of the target peptide, where the cleavable tag also includes an amino acid with a charged side-chain in the local environment of the tryptophan, such as within five amino acids on the upstream (i.e. amino) or downstream (i.e. carboxy) side of the tryptophan. In various embodiments, the presence of an amino acid side-chain within five amino acids on the amino terminus of the tryptophan amino acid allows for selectivity of cleavage of the tryptophan of the cleavable tag over any other tryptophans that may be present in the fusion peptide, for example, tryptophans as part of the inclusion body directing peptide or as part of the target peptide. For example, in various embodiments, an amino acid with a positively charged side chain such as lysine, ornithine, or arginine is within five, four, three, or two amino acid units, or is adjacent on the amino terminus to the tryptophan of the cleavable tag. In various embodiments, a glutamic acid amino acid is within five, four, three, or two amino acid units, or is adjacent on the amino terminus to the tryptophan of the cleavable tag.

In various embodiments, the cleavable tag is His-Met, or Pro-Met.

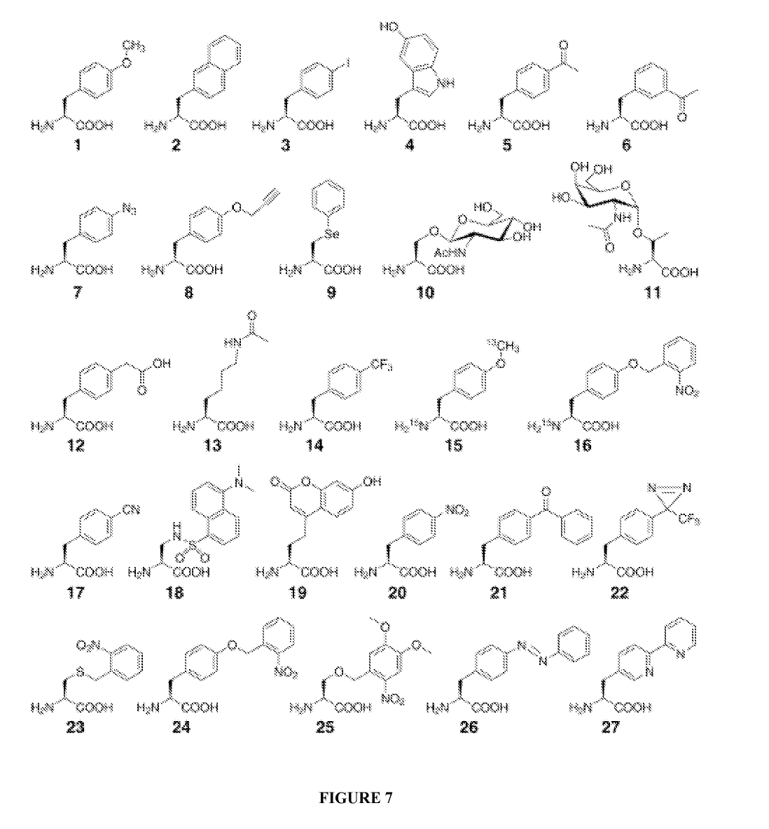

In various embodiments, the cleavable tag is an unnatural amino acid. Cells have been modified to enable the cells to produce peptides which contain unnatural amino acids. For instance, Wang, et al., (2001) Science 292:498-500, describes modifications made to the protein biosynthetic machinery of E. coli which allow the site-specific incorporation of an unnatural amino acid, O-methyl-L-tyrosine, in response to an amber stop codon (TAG). Wang, et al., (2009) Chem. Biol. 16(3):323-36 provides a review of numerous unnatural amino acids that have been site-specifically incorporated into proteins in E. coli, yeast, or mammalian cells. Without wishing to be bound by theory, it is believed that incorporation of one or more unnatural amino acids can provide additional selectivity for cleavage at the unnatural amino acid over non-specific cleavage at other sites on the fusion peptide. In various embodiments, the unnatural amino acid is selected from compounds I-27 in FIG. 7.

In some aspects, methods and compositions described herein include the production of fusion peptides comprising unnatural amino acids. In some aspects, prokaryotic cells with modifications to the protein biosynthetic machinery produce such fusion peptides. Examples of such prokaryotic cells include E. coli. In some aspects the modifications comprise adding orthogonal tRNA/synthetase pairs. In some aspects four base codons encode novel amino acids. In some aspects, E. coli allow the site-specific incorporation of the unnatural amino acid O-methyl-L-tyrosine into a peptide in response to an amber stop codon (TAG) being included in an expression vector.

### V. Fusion Peptide Synthesis

### A. Ribosomal Synthesis

In various embodiments, peptides may be produced by ribosomal synthesis, which utilizes the fundamental methods of transcription and translation to express peptides. Ribosomal synthesis is usually performed by manipulating the genetic code of various expression systems. Some peptides can be expressed in their native form in eukaryotic hosts such as Chinese hamster ovary (CHO) cells. Animal cell culture may require prolonged growing times to achieve maximum cell density and may achieve lower cell density than prokaryotic cell cultures (see Cleland, J. (1993) ACS Symposium Series 526, Protein Folding: In Vivo and In Vitro, American Chemical Society). Bacterial host expression systems such as Escherichia coli may achieve higher productivity than animal cell culture, and may have fewer regulatory hurdles for peptides intended to be used therapeutically. Numerous U.S. patents on general bacterial expression of recombinant proteins exist, including U.S. Pat. No. 4,565,785.

In one embodiment, the expression system is a microbial expression system. For example, in one embodiment, the process uses E. coli cells.

1. Construction of Vectors

In various embodiments, the method involves the construction of a DNA vector which includes certain selectable markers (such as antibiotic resistance in the case of E. coli) enabling selective screening against the cells that do not contain the constructed vector with the gene of interest. Vectors according to methods and compositions described herein may include hybrid promoters and multiple cloning sites for the incorporation of different genes. Various expression vectors may include the pET system and the pBAD system.

The pET system encompasses more than 40 different variations on the standard pET vector. In various embodiments, the pET system utilizes a T7 promoter that is recognized specifically by T7 RNA polymerase. This polymerase can transcribe DNA five times faster than E. coli RNA polymerase allowing for increased levels of transcription. In various embodiments, the Escherichia coli are protease deficient.

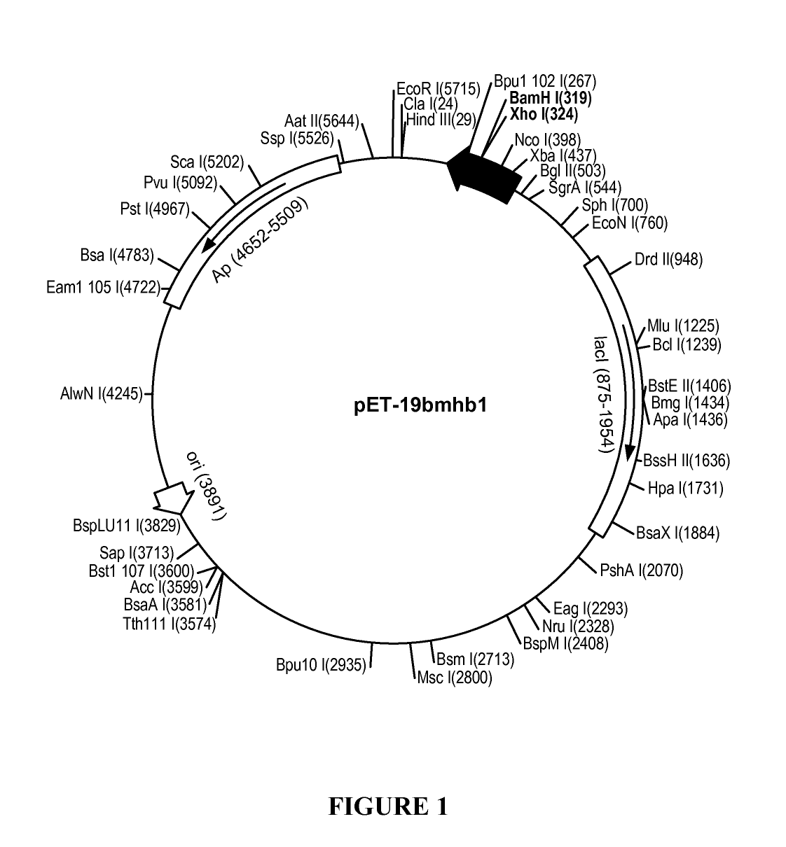

In one embodiment, a vector is designed with a sequences coding for a fusion peptide comprising an inclusion-body directing peptide, an affinity tag peptide, a cleavable peptide, and the target peptide. For example, in one embodiment, the vector is a pET-19b vector is modified to include a ketosteroid isomerase (KSI) sequence as the inclusion-body directing peptide. Thus, following cleavage of a restriction site such as the NcoI restriction site and insertion of the KSI sequence, the KSI sequence is flanked by two NcoI restriction sites. In addition, such a vector may be modified to include a histidine tag sequence as the sequence coding for an affinity tag adjacent to a tryptophan-encoding tag sequence as the sequence coding for a cleavable peptide which is further adjacent to a sequence coding for a target peptide such as the beta-amyloid (1-42) sequence. If an XhoI restriction site is used for purposes of insertion, the newly inserted sequence is flanked by two XhoI restriction sites. FIG. 1 diagrams one embodiment of a modified pET-19b (pET-19bmhb1) vector that can be used to produce a KSI sequence flanked by two NcoI restriction sites, and a histidine tag-tryptophan-beta-amyloid (1-42) sequence flanked by two XhoI restriction sites.

As such, a vector according to methods and compositions described herein such as a modified pET-19b vector contains the desired fusion peptide in a four part sequence: a KSI sequence or functional fragment to sequester the synthesized fusion protein into inclusion bodies, an affinity tag such as hexahistidine, a cleavage tag such as a tryptophan, and the target peptide.

2. Inoculation and Induction or Activation

Upon construction of an appropriate vector, the vector may be introduced into a host cell according to any method, and expression of the desired fusion peptide may be induced or activated by any method in the art.

In some embodiments once constructed, a vector according to methods and compositions described herein is inoculated or transformed into competent cells. In various embodiments, the competent cells may be mammalian cells such as Chinese hamster ovary cells, or microbial cells, such as E. coli cells. For example, the cells may be commercially available, such as DH5-ot E. coli cells (available from Invitrogen).

In various embodiments, transformed cells can be plated onto agar containing an antibacterial agent to prevent the growth of any cells that do not contain a resistance gene, thereby selecting for cells that have been transformed. In some embodiments, transformed E. coli cells are plated onto agar containing ampicillin to prevent the growth of any E. coli strains that do not contain the constructed pET-19b vector, and a colony is selected for further expansion.

Colonies from the plating process may be grown in starter culture or broth according to standard cell culture techniques. For example, in some embodiments, one colony from an agar plate is grown in a starter culture of broth, which may optionally contain an antibacterial agent. Typically, cells are grown to a preselected optical density before being further processed to obtain fusion peptide. For example, cells may be grown to an optical density (OD) of 0.1, 0.2, 0.3, 0.4, 0.5, 0.6, 0.7, 0.8, or 0.9, all values being about. In some embodiments the cells are grown to an optical density (OD) of about 0.5.

In a bacterial expression system, once the vector-containing bacterial cells have been isolated, inducible transcription may be used to produce the desired fusion peptide. For example, in E. coli cells, the lac operon serves as an inducible promoter that is activated under certain environmental conditions. E. coli are always capable of metabolizing the monosaccharide glucose. However, in order to metabolize the disaccharide lactose, the cells need an enzyme known as β-galactosidase. Thus, low extracellular glucose concentrations and high lactose concentrations induce the lac operon and the gene for β-galactosidase is transcribed. Accordingly, in various embodiments, an inducible promoter such as the lac operon is situated upstream from the sequence coding for the fusion peptide. Upon induction of the lac operon, transcription of the sequence coding for the desired fusion peptide occurs.

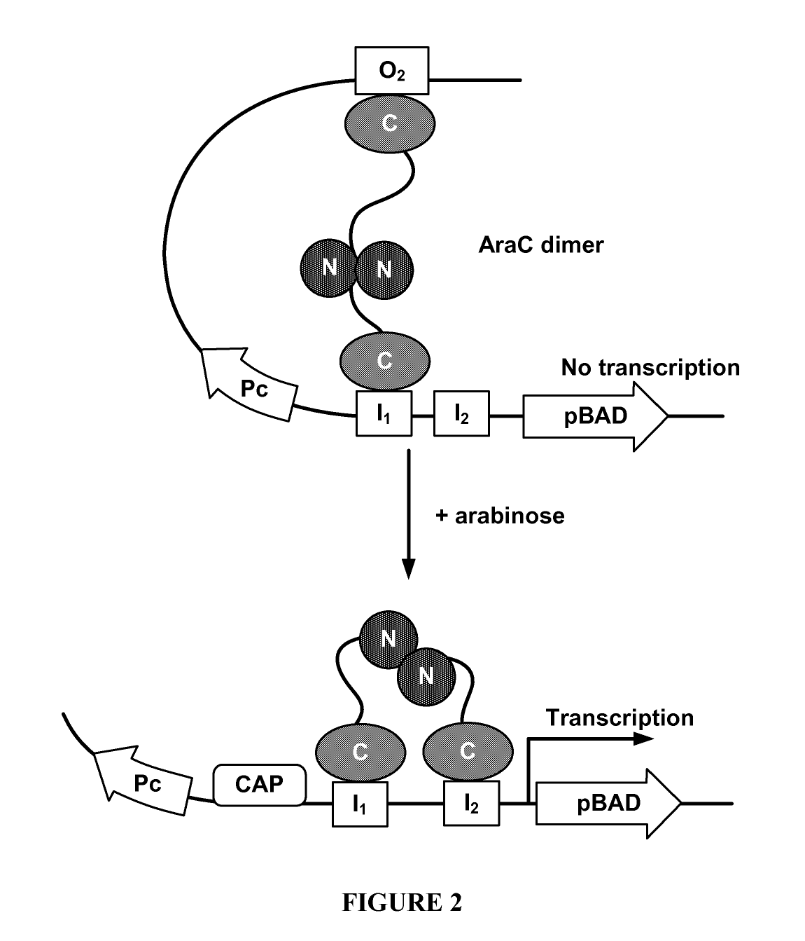

The term “activation” refers to the removal of repressor protein. A repressor protein is generally allosteric meaning it changes shape when bound by an inducer molecule and dissociates from the promoter. This dissociation allows for the transcription complex to assemble on DNA and initiate transcription of any genes downstream of the promoter. Therefore, by splicing genes produced in vitro into the bacterial genome, one can control the expression of novel genes. This trait may be used advantageously when dealing with inclusion bodies if the production and amassing of inclusion bodies becomes toxic enough to kill E. coli. For example, expression of the desired fusion peptide can be delayed until a sufficient population of cells has been cultured, and then the promoter can be induced to express a large amount of fusion peptide by removal of the repressor protein. Thus, the L-arabinose operon may be activated according to methods and compositions described herein for increased protein expression at a desired time point. Specifically, the L-arabinose operon may be activated by both the addition of L-arabinose into the growth medium and the addition of IPTG, a molecule that acts as an activator to dissociate the repressor protein from the operator DNA. FIG. 2 illustrates one embodiment of the activation of transcription in a pBAD vector via the addition of L-arabinose. Without wishing to be bound by theory, it is believed that L-arabinose binds to the AraC dimer causing the protein to release the O₂ site on the DNA and bind to the I₂ site. These steps serve to release the DNA loop and enable its transcription. Additionally, the cAMP activator protein (CAP) complex stimulates AraC binding to I_{I} and I₂—a process initiated with IPTG.

3. Targeting Expressed Peptides to Inclusion Bodies

In some cases, cells expressing only a fusion peptide with an affinity tag, a cleavable tag, and the target peptide cannot produce large amounts of fusion peptide. The reasons for low production yields may vary. For example, the fusion peptide may be toxic to the bacteria, thus causing the bacteria to die upon production of certain levels of the fusion peptide. Alternatively, the target peptide may be either poorly expressed or rapidly degraded in the bacterial system. In various scenarios, the target peptide may be modified by the host cell, including modifications such as glycosylation. To remedy some or all of these problems, the desired fusion peptide may be directed to an inclusion body, thereby physically segregating the target peptide from degradative factors in the cell's cytoplasm or, in the case of target peptides that are toxic to the host such as peptide antibiotics, physically segregating the target peptide to avoid toxic effects on the host. Moreover, by physically aggregating the fusion peptide in an inclusion body, the subsequent separation of the fusion peptide from the constituents of the host cell and the media (i.e., cell culture or broth) may be performed more easily or efficiently.

Target peptides may be directed to inclusion bodies by producing the target peptide as part of a fusion peptide where the target peptide is linked either directly or indirectly via intermediary peptides with an inclusion-body directing peptide. In various embodiments, an otherwise identical fusion peptide without an inclusion-body directing peptide has minimal or no tendency to be directed to inclusion bodies in an expression system. Alternatively, an otherwise identical fusion peptide without an inclusion-body directing peptide has some tendency to be directed to inclusion bodies in an expression system, but the number, volume, or weight of inclusion bodies is increased by producing a fusion peptide with an inclusion-body directing peptide. In various embodiments, where the target peptide itself directs the fusion peptide of the invention to inclusion bodies, a separate inclusion-body directing peptide may be excluded.

Any inclusion-body directing peptide may be used according to the methods of the invention. For example, methods have been described which allow α-human atrial natriuretic peptide (α-hANP) to be synthesized in stable form in E. coli. Eight copies of the synthetic α-hANP gene were linked in tandem, separated by codons specifying a four amino acid linker with lysine residues flanking the authentic N and C-termini of the 28 amino acid hormone. That sequence was then joined to the 3′ end of the fragment containing the lac promoter and the leader sequence coding for the first seven N terminal amino acids of β-galactosidase. The expressed multidomain protein accumulated intracellularly into stable inclusion bodies and was purified by urea extraction of the insoluble cell fraction. The purified protein was cleaved into monomers by digestion with endoproteinase lys C and trimmed to expose the authentic C-terminus by digestion with carboxypeptidase B. See Lennick et al., “High-level expression of α-human atrial natriuretic peptide from multiple joined genes in Escherichia coli,” Gene, 61:103-112 (1987), incorporated by reference herein.

In various embodiments, directing the target peptide to an inclusion body by producing the target peptide as part of a fusion peptide may lead to higher output of peptide. For example, in various embodiments, the desired fusion peptide is produced in concentrations greater than 100 mg/L. In various embodiments, the desired fusion peptide is produced in concentrations greater than about 200 mg/L, 250 mg/L, 300 mg/L, 350 mg/L, 400 mg/L, 450 mg/L, 500 mg/L, 550 mg/L, 600 mg/L, 650 mg/L, 700 mg/L, 750 mg/L, 800 mg/L, 850 mg/L, 900 mg/L, 950 mg/L, and 1 g/L, all amounts being prefaced by “greater than about.” In various embodiments, the output of desired fusion peptide is greater than about 1.5 g/L, greater than about 2 g/L, or greater than about 2.5 g/L. In various embodiments, the output of desired fusion peptide is in the range of from about 500 mg/L to about 2 g/L, or from about 1 g/L to about 2.5 g/L. In one embodiment, the desired fusion peptide is produced in yields equal to or greater than 500 mg/L of media.

In one embodiment, the inclusion-body directing peptide is a ketosteroid isomerase (KSI) or inclusion-body directing functional fragment thereof. In certain embodiments, inclusion-body directing functional fragment has at least 25, at least 30, at least 35, at least 40, at least 45, at least 50, at least 55, at least 60, at least 65, at least 70, at least 75, at least 80, at least 85, at least 90, at least 95, or at least 100 amino acids. Homologs of a ketosteroid isomerase are also encompassed. Such homologs may have at least 25, at least 30, at least 35, at least 40, at least 45, at least 50, at least 55, at least 60, at least 65, at least 70, at least 75, at least 80, at least 85, at least 90, or at least 95 percent sequence identity with the amino acid sequence of a ketosteroid isomerase. In various embodiments, an expression system for a fusion peptide with a functional fragment or homolog of a ketosteroid isomerase will produce at least 25, at least 30, at least 35, at least 40, at least 45, at least 50, at least 55, at least 60, at least 65, at least 70, at least 75, at least 80, at least 85, at least 90, at least 95, or greater than 100 percent of the amount of inclusion bodies produced by an otherwise identical expression system with a fusion peptide containing a complete ketosteroid isomerase peptide sequence.

4. Ribosomal Synthesis of Deuterated Peptides

Described herein are methods and compositions for producing deuterated peptides via ribosomal synthesis. In one embodiment, a deuterated peptide is synthesized by culturing a genetically modified host organism in a media containing deuterated amino acid. In another embodiment, a genetically modified host organism is a bacterium. In another embodiment, the bacterium is an E. coli. In another embodiment, the E. coli is a commonly used laboratory strain. An example of commonly used laboratory stain includes, but is not limited to, AG1, AB1157, BL21, BL21(AI), BL21(DE3), BL21 (DE3) pLysS, BL21(DE3)-CodonPlus-RIL™ BNN93, BNN97, BW26434, CGSC Strain #7658, C600, C600 hflA150 (Y1073, BNN102), CSH50, D1210, DB, DH1, DH5α, DH10B, DH12S, DM1, E. cloni(r) 5Alpha™, E. cloni(r) 10G™, E. cloni(r) 10GF′™, E. coli K12 ER2738™, ER2566™, ER2267™, HB101, HMS174(DE3), High-Control BL21(DE3), High-Control™ 10G, IJ1126, IJ1127, JM83, JM101, JM103, JM105, JM106, JM107, JM108, JM109, JM109(DE3), JM110, JM2.300, LE392, Mach1, MC1061, MC4100, MG1655, OmniMAX2™, OverExpress™ C41(DE3), OverExpress™ C41(DE3)pLysS, OverExpress™ C43(DE3), OverExpressC43™ (DE3)pLysS, Rosetta (DE3)pLysS, Rosetta-gami™ (DE3)pLysS, RR1, SOLR, SS320, STBL2, STBL3, STBL4, SURE, SURE2™, TG1, TOP10™, Top10F′™, W3110, XL1-Blue™, XL1-Blue MRF′™, XL2-Blue™, XL2-Blue MRF′™, XL1-Red™, and XL10-Gold™. In another embodiment, the host E. coli comprises one or more mutations in the host genome. An example of mutation includes, but is not limited to, F−, F+, F′[ ], rB/K+/−, mB/K+/−, hsdS, hsdR, INV( ), ahpC, ara-14, araD, cycA, dapD, Δ( ), dam, dcm, deoR, dnaJ, dutl, endA1, (e14), galE, galk, galU, gor, glnV, gyrA96, gyrA462, hflA150, Δ(lac)X74, lacIq or lacIQ, lacIQ1, lacY, lacZΔM15, leuB, Alon, malA, mcrA, mcrB, metB, metC, mrr, mtlA, (Mu), mutS, nupG, ompT, (P1), (P2), (φ80), pLysS, proA/B, recA1, recA13, recBCD, recJ, relA, rha, rnc, rne, rpsL, sbcBC, srl, supE, supF, thi, thyA, Tn10, Tn5, tonA, traD, trxB, tsx, tyrT, ungl, xyl-5, and SmR.

In another embodiment, the host microorganism is yeast. A strain of yeast may be selected from Saccharomyces cerevisiae, Saccharomyces pombe, a strain Pichia such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, or Pichia sp. In another embodiment, a yeast strain is a commonly used laboratory strain. Examples of laboratory yeast strain include, but are not limited to, S288C, 8Y4743, FY4, FY1679, AB972, A364A, XJ24-24a, DC5, YNN216, YPH499, YPH500, YPH501, Sigma 1278B, SK1, CEN.PK (aka CEN.PK2), W303-1A, W303-1B, X2180-1A, D273-10B, FL100, SEY6210, SEY6211, and JK9-3d, RM11-1a.

In another embodiment, the host organism is an insect cell. In another embodiment, a strain of insect cell may be selected from Spodoptera frugiperda. In another embodiment, a strain of insect cell may be commonly used laboratory strain, such as Sf9 or Sf21 cell. In another embodiment, the host organism is a mammalian cell.

In another embodiment, the host organism is a mammalian cell line. Examples of mammalian cell line include, but are not limited to, commonly used laboratory cell lines such as CHO cells, NIH3T3 cells, COS cells, or HeLa cells. In another embodiment, mammalian cell lines include, but are not limited to, laboratory cell lines commonly used for antibody production.

In one aspect, a medium for a host organism contains one or more deuterated amino acids. In one embodiment, deuterated amino acids are added to culture media commonly used to grow host strains described herein. In another embodiment, deuterated amino acids comprise a certain percentage of the total amount of amino acids in a medium. For example, deuterated amino acid may comprises about 1%, 2%, 4%, 8%, 9.5%, 11.8%, 14.1%, 16.4%, 18.7%, 21%, 23.3%, 25.6%, 27.9%, 30.2%, 32.5%, 34.8%, 37.1%, 39.4%, 41.7%, 44%, 46.3%, 48.6%, 50.9%, 53.2%, 55.5%, 57.8%, 60.1%, 62.4%, 64.7%, 67%, 69.3%, 71.6%, 73.9%, 76.2%, 78.5%, 80.8%, 83.1%, 85.4%, 87.7%, 90%, 92.3%, 94.6%, 96.9%, 99.2%, or 99.9% of the total amount of amino acids in a medium. In another embodiment, a medium may contain deuterated sugars such as C5 or C6 sugars. In another embodiment, a medium may contain deuterated lipids.

In one aspect, a heavy water (i.e., D₂O) is used to produce deuterated peptide. In one embodiment, a host organism described herein is cultured in a medium containing heavy water. In another embodiment, a peptide synthesized or isolated from a host organism is exposed to a solution containing heavy water. In another embodiment, an inclusion body is exposed to a solution containing heavy water. In another embodiment, the solution is buffered. In another embodiment, the solution comprises heavy water wherein the ratio of deuterium to hydrogen is greater than the ratio of deuterium to hydrogen in the peptide or inclusion body, and thereby facilitating deuterium-hydrogen exchange between heavy water and the peptide or inclusion body.

Described herein are methods of culturing host organism in a deuterated medium. The host organism may be cultured for a fixed duration of time without being monitored for its rate or growth. The host organism may be cultured while being monitored for its growth. In one embodiment, optical density (O.D₅₉₅) is measured periodically to monitor the growth of host organism. In another embodiment, a scintillation counter is used to monitor the rate of incorporation of deuterium. Where O.D. is used to monitor the growth, the O.D₅₉₅ may be about 0.2, 0.4, 0.6, 0.8, 1.0, 1.2, 1.4, 1.6, 1.8, or 1.95.

In one embodiment, the host organism is cultured in a bioreactor. In another embodiment, the bioreactor comprises a continuous feeding and harvesting system wherein the host organism may be removed from the bioreactor after satisfying a set of pre-determined culture parameters for deuterated medium. Examples of pre-determined culture parameters include, but are not limited to, pH, temperature, pressure of the bioreactor, concentration measured by optical density or other commonly used laboratory instruments, time, viscosity, morphology, and cell division rate.

In one embodiment, two different growth media are used to produce deuterated peptides in host organism. For example, a first growth medium does not contain deuterium while a second medium contains deuterium. In another embodiment, a first and third culture media do not contain deuterium while a second culture medium contains deuterium. A host organism may be grown in the first medium for a period of time and immediately switched to the second medium. A host organism may be grown in the first medium until it reaches an optical density indicating exponential growth phase and then switched to the second medium. A host organism grown on the first medium may be harvested, washed in a buffered solution, and then is resuspended for further culture in the second medium.

In one embodiment, one or more codons of a host organism are engineered to accommodate efficient deuteration and production of a target peptide. In one embodiment, codons frequently used in bacteria are modified to codons frequently used in mammal to efficiently deuterate and to produce mammalian peptide in a bacterial host organism. In some embodiments, codon usages of yeast are changed.

In one embodiment, peptides described herein are glycosylated. Glycosylated peptides are produced by employing host organisms capable of glycosylating peptides, such as insect or animal cells. In another embodiment, only carbohydrates attached to peptides described herein, but not amino acids of the peptides, are deuterated. In another embodiment, both carbohydrates and amino acids are deuterated. In another embodiment, amino acids, but not carbohydrates are deuterated.

### B. Solid Phase Peptide Synthesis

In one embodiment, the desired fusion peptide is made through solid phase peptide synthesis (SPPS). SPPS involves covalently linking a short peptide to an insoluble polymer providing a structural support for the elongation of the peptide. To achieve elongation of the peptide, the practitioner performs a series of repeated cycles de-protecting the chemically reactive portions of amino acids, linking the de-protected free terminal amine (N) to a single N-protected amino acid, de-protecting the N-terminal amine of the newly added residue, and repeating this process until the desired peptide has been built. Additional measures may be necessary for peptides that are about 50 or more amino acids in length.

In one embodiment, the solid phase peptide synthesis uses Fmoc protecting groups. The Fmoc protecting group utilizes a base labile alpha-amino protecting group. In an alternative embodiment, the solid phase peptide synthesis uses Boc protecting groups. The Boc protecting group is an acid labile alpha-amino protecting group. Each method may involve distinct resin addition, amino acid side-chain protection, and consequent cleavage/deprotection steps. Generally, Fmoc chemistry generates peptides of higher quality and in greater yield than Boc chemistry. Impurities in Boc-synthesized peptides are mostly attributed to cleavage problems, dehydration and t-butylation. Once assembled on the solid support, the peptide is cleaved from the resin using strongly acidic conditions, usually with the application of trifluoracetic acid (TFA). It is then purified using reverse phase high pressure liquid chromatography, or RP-HPLC, a process in which sample is extruded through a densely packed column and the amount of time it takes for different samples to pass through the column (known as a retention time) is recorded. As such, impurities are separated out from the sample based on the principle that smaller peptides pass through the column with shorter retention times and vice versa. Thus, the protein being purified elutes with a characteristic retention time that differs from the rest of the impurities in the sample, thus providing separation of the desired protein.

Solid-phase peptide synthesis generally provides high yields because excess reagents can be used to force reactions to completion. Separation of soluble byproducts is simplified by the attachment of the peptide to the insoluble support throughout the synthesis. Because the synthesis occurs in the same vessel for the entire process, mechanical loss of material is low.

In various embodiments, an inclusion body directing peptide may be excluded. Alternatively, an inclusion body directing peptide may be included to provide beneficial folding properties and/or solubility/aggregating properties.

In one aspect, peptides produced by solid-phase synthesis methods described herein are deuterated. In one embodiment, a peptide is catalytically deuterated using catalyst such as palladium oxide. In various embodiments, a peptide is dissolved in a suitable solvent such as water, dioxane, methanol, dimethlyformamide, benzene, toluene, or xylene. In various embodiments, the dissolved peptide is exposed to a catalyst in the presence of deuterium under pressurized condition. In various embodiments, the deuterium may be provided as charged gas. In various embodiments, the pressure may range from 0.1 to 100 atmospheres. In various embodiments, the catalysis reaction may last from hours to days. For example, the catalysis reaction may last for about 1, 2, 3, 4, 5, 6, 7, 8 or 12 hours. In another embodiment, the catalysis reaction may last for about a day. A peptide catalyzed by various processed described herein can be filtered to purity by filtering off the catalyst. In some embodiment, the filtered peptide is washed in an appropriate buffer solution. In some embodiments, washing comprises dialysis and re-concentration. In some embodiments, the filtered and deuterated peptide is dried. In some other embodiments, the filtered and deuterated peptide is lyophilized.

### C. Non-Ribosomal Synthesis

In various embodiments, peptides may be produced by non-ribosomal synthesis. Such peptides include circular peptides and/or depsipeptides.

Nonribosomal peptides are synthesized by one or more nonribosomal peptide synthetase (NRPS) enzymes. These enzymes are independent of messenger RNA. Nonribosomal peptides often have a cyclic and/or branched structure, can contain non-proteinogenic amino acids including D-amino acids, carry modifications like N-methyl and N-formyl groups, or are glycosylated, acylated, halogenated, or hydroxylated. Cyclization of amino acids against the peptide backbone is often performed, resulting in oxazolines and thiazolines; these can be further oxidized or reduced. On occasion, dehydration is performed on serines, resulting in dehydroalanine.

The enzymes of an NRPS are organized in modules that are responsible for the introduction of one additional amino acid. Each module consists of several domains with defined functions, separated by short spacer regions of about 15 amino acids. While not wishing to be bound by theory, it is thought that a typical NRPS module is organized as follows: initiation module, one or more elongation modules, and a termination module. The NRPS genes for a certain peptide are usually organized in one operon in bacteria and in gene clusters in eukaryotes.

In one embodiment, a deuterated peptide is produced via NRPS-mediated pathway in a host organism. In another embodiment, a host organism is a bacteria or a fungus. In another embodiment, a host organism is culture in a medium containing deuterated metabolites or nutrients. Deuterated metabolites or nutrients include, but are not limited to, deuterated fatty acids, polyketides, ATP, serine, threonine, cysteine, oxazolidines, thazolidines, alcohol, acyl-CoA, or acetate.

In various embodiments, an inclusion body directing peptide may be excluded. Alternatively, an inclusion body directing peptide may be included to provide beneficial folding properties and/or solubility/aggregating properties.

VI. Separation of Fusion Peptide from Formation Media

Following production of the desired fusion peptides, separation from the production media is required. Optionally, following separation, the desired fusion peptide and carrier may be concentrated to remove excess liquid. Numerous methods for separating fusion peptides from their formation media and subsequent handling may be adapted to the invention. Various methods are described in detail herein.

In some embodiments, desired fusion peptides described herein are deuterated peptides. In some embodiments, deuterated peptides are separated from the formation media or host organism in a substantially similar manner to non-deuterated peptides. In some embodiments, deuterated peptides are separated from the formation media or host organism in the same manner as applied to non-deuterated peptides other than performing minor modifications in the separation methods necessary to comply with relevant safety regulations on handling isotopic material.

A. Fusion Peptides Targeted to Inclusion Bodies

In various embodiments, the cells used to produce the desired fusion peptides may be lysed to release the fusion peptides. For example, where the desired fusion peptide is aggregated in inclusion bodies, the cell may by lysed, followed by separation of the inclusion bodies from the production media and cellular detritus. Any method of cell lysis may be used.

In various embodiments, cells are disrupted using high-power sonication in a lysis buffer. For example, a lysis buffer may be added before lysis containing Tris, sodium chloride, glycerol, and a protease inhibitor. In one embodiment, a lysis buffer containing about 25 mM Tris pH 8.0, about 50 mM NaCl, about 10% glycerol, and the protease inhibitor 1000×PMSF may be added before lysis. Insoluble inclusion bodies may be collected using one or more washing steps and centrifugation steps. Wash buffers may include any reagents used for the stabilization and isolation of proteins. For example, in various embodiments, wash buffers are used containing varying concentrations of Tris pH 8.0, NaCl, and Triton X100.

In one embodiment, targeting the desired fusion peptide to an inclusion body may result in higher initial purity upon lysis of the cell. For example, in one embodiment, lysis of the cell and isolation of inclusion bodies through physical means such as centrifugation may result in an initial purity of greater than about 70%, great than about 75%, greater than about 80%, greater than about 85%, greater than about 90%, or greater than about 95% for the desired fusion peptide.

In some embodiments following cell lysis, inclusion bodies form a pellet and remain in the pellet rather than supernatant until a solubilization step. In various embodiments, the pellet is washed clean of the remaining cellular components, and insoluble inclusion bodies are solubilized in a buffer for further handling. Solubilization buffers may include urea or any other chaotropic agent necessary to solubilize the fusion peptide. Without wishing to be bound by theory, it is believed that the solubilization step involves solubilizing the inclusion bodies in a chaotropic agent which serves to disrupt the peptides by interfering with any stabilizing intra-molecular interactions.

In various embodiments, the solubilization buffer may include urea, guanidinium salts, or organic solvents. For example, a solubilization buffer may contain about 25 mM Tris pH 8.0, about 50 mM, NaCl, about 0.1 mM PMSF, and about 8M urea. In some embodiments, solubilization of inclusion bodies occurs with the addition of 8M urea as the sole chaotropic agent, and other chaotropic agents are excluded. Alternatively, the solubilization buffer may exclude urea or guanidinium salts. For example, in one embodiment, guanidinium salts are excluded to avoid interference with further processing on an ion exchange column. As an additional example, in one embodiment, high urea concentrations such as about 8M urea are excluded to avoid denaturing proteases that may be included in the solubilization buffer.

In various embodiments, a minimal amount of solubilization buffer is used. In the event that excess solubilization buffer is present, the solution may be processed to remove excess solvent prior to further purification.

B. Fusion Peptides Not Targeted to Inclusion Bodies

In various embodiments, fusion peptides are not directed to inclusion bodies. In such embodiments, the fusion peptides may remain in the cytosol of the cell, or the fusion peptides according to methods and compositions described herein may be secreted from the cell. In one embodiment, the secretion may comprise a budding process. In another embodiment, the secretion may comprise active transport of the fusion peptide via exocytosis. In another embodiment, the secreted peptide comprises signaling peptide directing the fusion peptide to secretion. In another embodiment, the fusion peptide may be targeted to membrane. In another embodiment, the membrane portion of a host organism may be harvested for further purification of the fusion peptides. Methods for isolating the membrane fraction from a host organism are known in the art. See Sambrook et al., Molecular Cloning: A Laboratory Manual, Cold Spring Harbor Press, 3rd Edition, 2001. Soluble fusion peptides may be isolated by any method, such as centrifugation, gel electrophoresis, pH or ion exchange chromatography, size exclusion chromatography, reversed-phase chromatography, dialysis, osmosis, filtration, and extraction.

### VII. Purification by Affinity Chromatography

Following cell lysis and initial isolation and solubilization of fusion peptides according to methods and compositions described herein, the fusion peptides are further purified by affinity chromatography, which is a highly selective process that relies on biologically-relevant interactions between an immobilized stationary phase and the fusion peptide to be purified. In various embodiments, the immobilized stationary phase is a resin or matrix. Without wishing to be bound by theory, it is believed that affinity chromatography functions by selective binding of the desired component from a mixture to the immobilized stationary phase, followed by washing of the stationary phase to remove any unbound material.

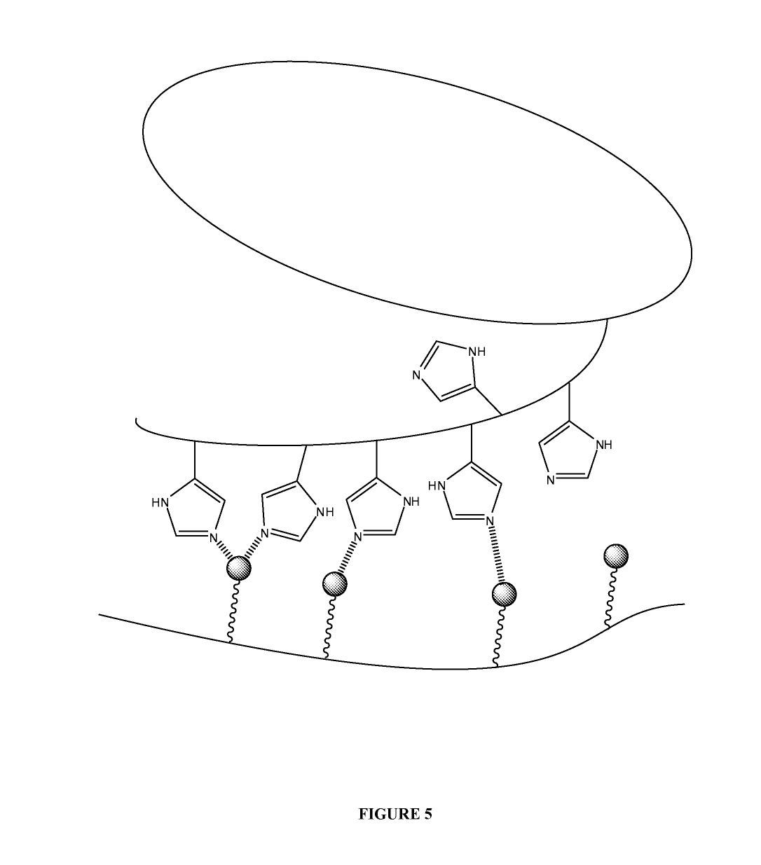

According to methods and compositions described herein, a wide variety of affinity chromatography systems may be used. For example, polyhistidine binds with great affinity and specificity to nickel and thus an affinity column of nickel, such as QIAGEN nickel columns, can be used for purification. See, e.g., Ausubel et al., eds., Current Protocols in Molecular Biology 10.11.8 (John Wiley & Sons 1993). Alternatively, Ni-NTA affinity chromatography resin (available from Invitrogen) may be used. FIG. 5 provides a schematic of an example of an immobilized Ni-NTA resin binding to a 6×HisTag on a protein. Metal affinity chromatography has been used as a basis for protein separations. See Arnold, “Metal Affinity Separations: A New Dimension In Protein Processing” Bio/Technology, 9:151-156 (1991). See also Smith et al., “Chelating Peptide-immobilized Metal Ion Affinity Chromatography” J. Biol. Chem., 263:7211-7215 (1988), which describes a specific metal chelating peptide on the NH₂ terminus of a protein that can be used to purify that protein using immobilized metal ion affinity chromatography.

According to methods and compositions described herein, the affinity column is equilibrated with buffer which may be the same as used for the solubilization of the fusion peptide. The column is then charged with the solubilized fusion peptide, and buffer is collected as it flows through the column. In various embodiments, the column is washed successively to remove urea and/or other impurities such as endotoxins, polysaccharides, and residual contaminants remaining from the cell expression system.

VIII. Removal of Target Peptide from Affinity Column via Cleavage

Described herein are numerous methods for cleavage of the fusion peptides on the affinity column. In general, the cleavage step occurs by introduction of a cleavage agent which interacts with the cleavage tag of the fusion peptide resulting in cleavage of the fusion peptide and release of the target peptide. Following cleavage, the affinity column may be flushed to elute the target peptide while the portion of the fusion peptide containing the affinity tag remains bound to the affinity column. Following elution of the target peptide, the eluting solution may be condensed to a desired concentration. The target peptide may be further processed and/or packaged for distribution or sale.

Control of the cleavage reaction may occur through chemical selectivity. For example, the cleavage tag may include a unique chemical moiety which is absent from the remainder of the fusion peptide such that the cleavage agent selectively interacts with the unique chemical moiety of the cleavage tag.

In various embodiments, control of the cleavage reaction occurs through a unique local environment. For example, the cleavage tag may include a chemical moiety that is present elsewhere in the fusion peptide, but the local environment differs resulting in a selective cleavage reaction at the cleavage tag. For example, in various embodiments, the cleavage tag includes a tryptophan and a charged amino acid side chain within five amino acids of the tryptophan. In various embodiments, the charged amino acid is on the amino terminus of the tryptophan amino acid.

In various embodiments, control of the cleavage reaction may occur through secondary or tertiary structure of the fusion peptide. For example, in various embodiments, where identical moieties are present in the cleavage tag and elsewhere in the fusion peptide, the other portions of the fusion peptide may fold in secondary or tertiary structure such as alpha-helices, beta-sheets, and the like, to physically protect the susceptible moiety, resulting in selective cleavage at the cleavage tag.

In various embodiments, minor or even major differences in selectivity of the cleavage reaction for the cleavage tag over other locations in the fusion peptide may be amplified by controlling the kinetics of the cleavage reaction. For example, in various embodiments, the concentration of cleavage agent is controlled by adjusting the flow rate of eluting solvent containing cleavage agent. In various embodiments, the concentration of cleavage agent is maintained at a low level to amplify differences in selectivity. In various embodiments, the reservoir for receiving the eluting solvent contains a quenching agent to stop further cleavage of target peptide that has been released from the column.

Moreover, various methods for removal of peptides from affinity columns may be excluded. For example, in some embodiments, the steps of removal may specifically exclude the step of washing an affinity column with a solution of a compound with competing affinity in the absence of a cleavage reaction. In one embodiment, the step of washing an affinity column with a solution of imidazole as a displacing agent to assist in removing a fusion peptide from an affinity column is specifically excluded. The concentration of imidazole may vary. For example, the concentration of imidazole to wash the column may include about 1-10 mM, 5-20 mM, 10-50 mM, 30-70 mM, 50-100 mM, 80-200 mM, 100-300 mM, 150-500 mM. Imidazole may be applied as a fixed concentration or as a gradient between two fixed concentration representing the lower and the upper limits. For example, a gradient of imidazole may be used to wash the column, starting from 1 mM and ending with 500 mM over a period of time.

In various embodiments, multiple cleavages are envisioned. For example, insulin is known to be produced from a proinsulin precursor requiring two cleavage events. Both cleavage events are required in order for the mature insulin to be properly folded. Accordingly, in various embodiments, a cleavage process may include two cleavage tags. Preferably, when more than one cleavage tag is present, the distinct cleavage tags are orthogonal, or able to be cleaved with specificity by different cleavage agents. For example, in one embodiment, one cleavage tag is a methionine amino acid while the other cleavage tag is a tryptophan amino acid.

In various embodiments, the cleavage agent is selected from the group consisting of NBS, NCS, cyanogen bromide, Pd(H₂O)₄, 2-ortho iodobenzoic acid, DMSO/sulfuric acid, or a proteolytic enzyme. Various methods and cleavage agents are described in detail herein.

A. NBS Cleavage

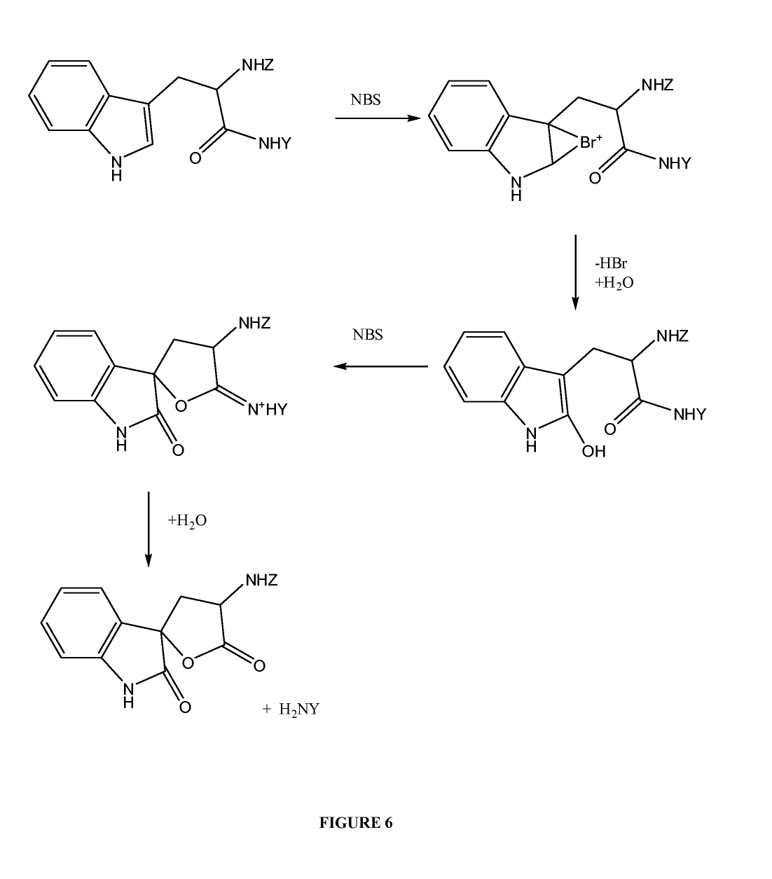

In one embodiment, the cleavage reaction according to methods and compositions described herein involves the use of a mild brominating agent N-bromosuccinimde (NBS) to selectively cleave a tryptophanyl peptide bond at the amino terminus of the target peptide. Without wishing to be bound by theory, it is believed that in aqueous and acidic conditions, NBS oxidizes the exposed indole ring of the tryptophan side chain, thus initiating a chemical transformation that results in cleavage of the peptide bond at this site. FIG. 6 illustrates one possible mechanism for the selective cleavage of tryptophan peptide bonds with N-bromosuccinimde. According to the mechanism, the active bromide ion halogenates the indole ring of the tryptophan residue followed by a spontaneous dehalogenation through a series of hydrolysis reactions. These reactions lead to the formation of an oxindole derivative which promotes the cleavage reaction.

B. NCS Cleavage

In one embodiment, the cleavage reaction according to methods and compositions described herein involves the use of a mild oxidizing agent N-chlorosuccinimde (NCS) to selectively cleave a tryptophanyl peptide bond at the amino terminus of the target peptide. Without wishing to be bound by theory, it is believed that in aqueous and acidic conditions, NCS oxidizes the exposed indole ring of the tryptophan side chain, thus initiating a chemical transformation that results in cleavage of the peptide bond at this site.

C. Enzymatic Cleavage

In various embodiments, enzymes may be employed to cleave the fusion protein. For example, Protease use is diverse yet selective as there are many proteases that recognize specific amino acid sequences. In various embodiments, the active site of a serine or threonine protease will bind to either serine or threonine, respectively, and initiate catalytic mechanisms that result in proteolysis. Additional enzymes include collagenase, enterokinase factor X_{A}, thrombin, trypsin, clostripain and alasubtilisin. See Uhlen and Moks, Meths. in Enz., 185:129-143 (1990) and Emtage, “Biotechnology & Protein Production” in Delivery Systems for Peptide Drugs, pp. 23-33 (1986).

D. Additional Chemical Agents

In various embodiments, the cleavage agent is a chemical agent such as cyanogen bromide, palladium (II) aqua complex (such as Pd(H₂O)₄), formic acid, and hydroxylamine. For example, cyanogen bromide may be used to selectively cleave a fusion peptide at a methionine amino acid at the amino terminus of the target peptide.

### IX. Downstream Processing

In various embodiments, target peptides produced according to the process described herein may be further modified. For example, in various embodiments, the C-terminus of the target peptide is connected to alpha-hydroxyglycine. At the desired time, the target peptide, either as the isolated target peptide or as part of the fusion peptide, is exposed to acid catalysis to yield glycolic acid and a carboxamide group at the carboxy terminus of the target peptide. A carboxamide group at the carboxy terminus is present in a variety of neuropeptides, and is thought to increase the half-life of various peptides in vivo.

In various embodiments, target peptides produced according to methods and compositions described herein may be further modified to alter in vivo activity. For example, in various embodiments, a polyethylene glycol (PEG) group may be added to a target peptide.

### X. Peptide Marketing

Described herein are methods directed to marketing the target peptides. In one embodiment, the commercial market for a target peptide is evaluated. Evaluative methods may include, but are not limited to, producing a target peptide as described herein, making sample amounts of the target peptide available for no cost or for minimal cost, and measuring the number of requests for the target peptide over a period of time. Advantages of making a target peptide available in this manner may include an improved calculation of the future supplies needed and/or future demand by paying customers. Alternatively, providing a target peptide at no cost or minimal cost initially may induce interest in the target peptide and the discovery of favorable characteristics for the peptide that spur future sales. Minimal cost may include a price that is approximately the cost of production with essentially no profit involved. In various embodiments, the minimal cost may be about 10%, about 20%, about 30%, about 40%, about 50%, about 60%, or about 70% of the price of a competitor's product.

In one aspect, deuterated or non-deuterated peptide described herein is provided as a kit. In one embodiment, a kit comprises deuterated peptide and non-deuterated peptide. In another embodiment, a kit comprises deuterated amino acids, a vector, a host organism, and an instruction manual. In another embodiment, a kit comprises deuterated water, a vector, a host organism, and an instruction manual. In another embodiment, a kit comprises deuterated amino acids, a vector, a host organism, a Ni+ column, imidazole, and an instruction manual. In another embodiment, a kit comprises an instruction manual describing methods and compositions disclosed herein.

### XI. Applications

Deuterated peptides described herein may be used for various applications. In one embodiment, the peptides may be used for laboratory experiments. Laboratory experiments include, but are not limited to, animal experiment, in vitro experiment such as protein-protein binding experiment, mapping active site of an enzyme or residues participating in an interaction between a particular pair of biological molecules, protein structural studies, identification of metabolic pathways, and quantitation experiments. In another embodiment, the peptides may be used for clinical purposes such as clinical diagnosis, treatment, prognosis, monitoring, and clinical trial. In another embodiment, the peptides maybe used for pharmacokinetics studies, pharmacodynamic studies, or other pharmacological and/or drug studies investigating absorption, digestion, metabolism, and excretion. In another embodiment, the peptides maybe used for marketing researches comparing the dollar amount spent on a particular therapy employing either a deuterated peptide or a non-deuterated peptide. In another embodiment, the peptides maybe used for drug efficacy testing. In another embodiment, the peptides maybe used for studies exploring off-label indications. In another embodiment, the peptides maybe used for veterinary purposes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

The examples herein provide some non-limiting examples according to methods and compositions described herein. The following examples include both actual examples and prophetic examples.

### Example 1

Cells are induced to initiate the synthesis of KSI-Abeta (1-42) with 1 mM IPTG (Invitrogen) and 0.2% L-arabinose (Calbiotech) as follows. Plated cells are incubated overnight at 37° C. and then one colony from this plate is grown up overnight in a starter culture of 8 mL of Luria broth+ampicillin. The following morning, the starter culture is inoculated into 1 L of Luria broth+ampicillin and grown to an optical density (OD) of 0.5. At this point, the cells are induced with 1 mM IPTG (Invitrogen) and 0.2% L-arabinose (Calbiotech) to initiate the synthesis of KSI-Abeta (1-42).

To optimize the amount of KSI-Abeta (1-42) production in the bacteria, samples of the 1 L inoculation are taken prior to inducing the bacteria, and then 2, 4, 6, and 16 hours (overnight growth) after induction. A 12% acrylamide gel is used to analyze the samples since the fusion protein weighs approximately 21 kD. Optimal fusion protein synthesis occurs when the culture is induced and grown overnight.

Eight hours after induction, the cells are re-induced with the same concentrations of IPTG and L-arabinose as well as 100 mg of ampicillin as to prevent the growth of any new strains of E. coli.

### Example 1A

The construct is re-designed to place a His-tag upstream from the KSI sequence rather than downstream.

### Example 2

Following induction of KSI-Abeta (1-42) production in E. coli, lysis buffer containing 25 mM Tris pH 8.0, 50 mM NaCl, 10% glycerol, and the protease inhibitor 1000×PMSF is added before lysis. Insoluble inclusion bodies are collected using washing and centrifugation. Three different wash buffers are used containing varying concentrations of Tris pH 8.0, NaC 1, and Triton X100. Once washed clean of the remaining cellular components, the insoluble inclusion bodies are solubilized in a buffer containing 25 mM Tris pH 8.0, 50 mM, NaCl, 0.1 mM PMSF, and 8M urea. The 8M urea serves as a chaotropic agent necessary in solubilizing protein.

A 12% acrylamide gel is run on both uninduced and induced bacteria, the cell lysate produced from high output sonication, and the supernatant from each washing step during the inclusion body preparation. The gel is stained with Coomassie Blue reagent. The appearance of a 21 kD in the induced sample provides evidence for inclusion body synthesis resulting from induction. Exemplary data shows the stages of inclusion body preparation by gel electrophoresis of cells lysed with high-power sonication and washed with a series of buffers containing different concentrations of Tris, NaCl, PMSF, Triton-X100, and urea. The disappearance of the 21 kD band during successive steps and reappearance of the 21 kD band upon solubilizing the inclusion bodies (lane 10) indicates that the inclusion bodies are properly prepared. Accordingly, a lane containing the cell lysate is almost entirely blue because as the cells are ruptured, relatively large quantities of various proteins are extracted. As the lysate is washed repeatedly of impurities, the lanes become clearer.

### Example 3

The concentration of protein in solubilized inclusion bodies is determined via a Bradford Assay. A series of NBS cleavage reactions is run to determine the optimal conditions for tryptophanyl peptide bond cleavage. Three concentrations of NBS purchased from TCI America (equimolar, 3×, and 6×) are allowed to react with KSI-Abeta (1-42) for varying amounts of time (0, 15, and 30 minutes) before being quenched with excess N-acetylmethionine (Acros). Since the amyloid beta cleavage product weighs only 5 kD, a higher percentage acrylamide gel (18%) is used to determine the success of the NBS cleavage in solution. The gel indicates that optimal cleavage occurs when 6×NBS is reacted with KSI-Abeta (1-42) at room temperature from 0 to 30 minutes. Exemplary data for gel electrophoresis shows nine different NBS cleavage reactions. The samples are run on an 18% acrylamide gel and silver stained. Lane (1) stock inclusion bodies; lane (2) OX NCS for 0 min; lane (3) 1×NBS for 30 min; lane (4) 1×NBS for 60 min; lane (5) 3×NBS for 0 min; lane (6) 3×NBS for 30 min; lane (7) 3×NBS for 60 min; lane (8) 6×NBS for 0 min; lane (9) 6×NBS for 30 min; lane (10) 6×NBS for 60 min.

### Example 4

Ni-NTA Affinity Chromatography resin purchased from Invitrogen is equilibrated with the same solubilization buffer as in the inclusion body preparation. Next, the resin is charged with the solubilized inclusion bodies and the flow through is collected. The column is then washed with five column volumes of 50% ethanol to remove urea and flow through. Afterwards, 3×NBS is loaded and the column is placed on a rocker for 30 minutes. At this time, the reaction is quenched with excess N-acetylmethionine and the flow through is collected. The column is then washed with 300 mM imidazole to discharge the remaining fusion protein and the flow through is collected.

SDS-PAGE analysis indicates that a very small amount of inclusion bodies adhere to the Ni-NTA column as evidenced by the appearance of a large 21 kD band in the first wash. Exemplary data for gel electrophoresis following Ni-NTA affinity chromatography is as follows. Inclusion bodies are loaded onto an equilibrated Ni-NTA column and washed with the same buffer, collecting the flow-through (lane 1). The column is then washed with 50% ethanol as to equilibrate it with the cleavage solution buffer (lane 2). On-column cleavage is performed with 3×NBS for 30 minutes at room temperature and the flow through is collected (lane 3). The column is washed with 300 mM imidazole to wash off all remaining fusion protein and the flow-through is collected (lane 4). A narrower band appears after the second wash in ethanol to equilibrate the column for the on-column cleavage. A very minor amount of cleavage does occur on the remaining KSI-Abeta (1-42). Incubating the inclusion bodies overnight on a rocker does not improve on-column cleavage, although it does improve the initial binding of KSI-Abeta (1-42) to the column.

### Example 5

SDS-PAGE analysis on the NBS solution cleavage indicates that the cleavage is successful in solution. Exemplary data shows gel electrophoresis of inclusion bodies that are reacted with 3×NBS for 30 min and then quenched with N-acetylmethionine. The same sample is loaded in increasing quantities (from 10 to 25 ml) to show appearance of 5 kD cleavage product.

Because optimal cleavage rates range from 35-45 percent, only a small amount of KSI-Abeta (1-42) is produced. Since the gel contains a small amount of diluted sample, the assay does not detect the 5 kD cleavage product with Coomassie Blue staining. Therefore, visualizing the cleavage product requires overloading the sample and overdeveloping the silver stain.

### Example 6

The manufacturing cost analysis of direct materials used indicates that the cost to synthesize beta-Amyloid (1-42) through a combination of recombinant expression, chemical manipulation, and purification is drastically lower than the prices charged by other manufacturers (noting that a large makeup of their prices contains overheads, operating costs, labor costs, etc.). Assuming an average yield of inclusion bodies for a 1 liter culture is approximately 5 grams per liter and a 50% NBS cleavage rate, as well as considering product lost during purification, an estimated $0.11 in materials is all that is necessary to synthesize 1 mg of beta-Amyloid (1-42). Even after factoring all of the other manufacturing costs such as facilities, equipment, and labor into the price, the synthesis of beta-Amyloid (1-42) using this method could cost much less to the consumer.

### Example 7

A nucleotide sequence of human beta-amyloid (1-42) peptide is obtained from publicly available genomic database. Based on the sequence information, PCR primers are designed to isolated cDNA sequences corresponding to the transcript of human beta-amyloid (1-42) gene. PCR is performed on a collection of cDNA derived from a population of RNAs containing transcripts for beta-amyloid. Alternatively, nucleic acid sequence for beta-amyloid is synthesized. In designing the PCR primers, the primers are flanked by appropriate sequences for endonucleases. The obtained PCR product is then cloned into a vector containing KSI sequence. The PCR product is cloned at the 3′ end of KSI sequence. After the cloning, the vector is sequenced to confirm the reading frame and to ensure correct translation of the 5′ affinity tag, KSI sequence, cleavage sequence, and beta-amyloid (1-42) sequence in that order. The vector containing correct sequence is selected and purified. The purified vector is transformed in E. coli BL21 (DE3). The E. coli is cultured overnight (about 12-18 hours) in 5 ml of E. coli culture medium. On the following day, the 5 ml confluent culture is inoculated in 500 ml of culture medium. The culture is continued at 37° C. with periodically checking the O.D. of the culture. When the O.D. reaches 0.2, the culture is stopped, harvested by centrifugation, and resuspended in a medium containing deuterated amino acids. The culture is resumed in a 37° C. incubator for 20 min. After this step, IPTG is added to the final concentration of 1 mM. The induction continues until O.D. reaches 0.6. Alternatively, the culture is stopped at O.D. 0.4 and continues until O.D. 0.8. The culture is harvested by centrifugation and the pellet is washed by resuspending the pellet in phosphate buffered saline and recentrifuged. The washed pellet is lysed with a Tris-HCl buffer containing proteinase K at 4° C. for 30 min. The cell lysate is vortexed and sonicated to shear genomic DNAs in the lysate. The lysate is then centrifuged and insoluble fraction is retained. The insoluble fraction is treated in a buffer containing 8M urea to solubilize inclusion bodies in the insoluble fraction. After solubilization, a centrifugation is performed to isolate supernatant from insoluble pellet. The supernatant is dialyzed to remove urea and exchange the buffer to Ni+ column running buffer containing 1 mM imidazole. To reduce the processing volume of the supernatant, the dialyzed supernatant is concentrated by filtering. The Ni+ column is equilibrated with 1 mM imidazole. After equilibration, the dialyzed and concentrated supernatant is loaded to the column. The column is washed with imidazole-containing buffer by slowly increasing the concentration of imidazole from 1 mM to 50 mM. The wash is repeated twice. After washing, NBS cleavage is performed within the column, and deuterated target peptide is eluted from the column. The identity of deuterated peptide is confirmed by peptide sequencing. The purity of deuterated peptide is confirmed on SDS-PAGE and by mass spectrometry.

While preferred embodiments have been shown and described herein, it will be apparent to those skilled in the art that such embodiments are provided by way of example only. Numerous variations, changes, and substitutions will now occur to those skilled in the art without departing from compositions and methods described herein. It should be understood that various alternatives to the embodiments of the invention described herein may be employed in practicing compositions and methods described herein.

What is claimed is:

1. A method for producing a deuterated target peptide comprising
a) producing a deuterated fusion peptide comprising an affinity tag, a cleavable tag, and the target peptide wherein said deuterated fusion peptide is deuterated at least in the region encompassing the target peptide;
b) binding said fusion peptide to an affinity material;
c) cleaving said fusion peptide to release the target peptide; and
d) removing the target peptide from the affinity material to yield a deuterated target peptide.

2. The method of claim 1, wherein said target peptide is selected from the group consisting of amyloid beta, calcitonin, enfuvirtide, epoetin, epoetin delta, erythropoietin, exenatide, factor VIII, factor X, glucocerebrosidase, glucagon-like peptide-1 (GLP-1), granulocyte-colony stimulating factor (G-CSF), human growth hormone (hGH), insulin, insulin A, insulin B, insulin-like growth factor 1 (IGF-1), interferon, liraglutide, somatostatin, teriparatide, and tissue plasminogen activator (TPA).

3. The method of claim 1, wherein said step of producing a fusion peptide is performed in a bacterial expression system comprising deuterium-containing culture medium.

4. The method of claim 3, wherein said deuterium-containing culture medium contains at least one amino acid or amino acid precursor with at least one non-exchangable hydrogen replaced with deuterium.

5. The method of claim 3, wherein said fusion peptide further comprises an inclusion-body directing peptide.

6. The method of claim 5, wherein prior to binding said fusion peptide to an affinity material, said method further comprises removal of inclusion bodies containing the fusion peptide from the bacterial expression system and solubilization of the fusion peptide in the inclusion bodies.

7. The method of claim 5, wherein said inclusion-body directing peptide is selected from the group consisting of inclusion-body directing peptide is a ketosteroid isomerase, an inclusion-body directing functional fragment of a ketosteroid isomerase, an inclusion-body directing functional homolog of a ketosteroid isomerase, a BRCA2 peptide, an inclusion-body directing functional fragment of BRCA2, or an inclusion-body directing functional homolog of BRCA2.

8. The method of claim 1, wherein subsequent to binding said deuterated fusion peptide to affinity material, said method further comprises washing the affinity material to remove unbound material.

9. The method of claim 1, wherein said affinity tag is selected from the group consisting of poly-histidine, poly-lysine, poly-aspartic acid, or poly-glutamic acid.

10. The method of claim 1, wherein said cleavable tag is selected from the group consisting of Trp, His-Met, Pro-Met, and an unnatural amino acid.

11. The method of claim 1, wherein said cleaving step is performed with an agent selected from the group consisting of NBS, NCS, or Pd(H₂O)₄.

12. A deuterated target peptide produced according to the method of claim 1, wherein said peptide is greater than 99% pure.

13. A deuterated target peptide produced according to the method of claim 1, wherein said peptide has at least 1% of its non-exchangable hydrogens replaced with deuterium.

14. A deuterated target peptide produced according to the method of claim 1, wherein said peptide has at least one amino acid that is labeled with deuterium.

15. A deuterated target peptide produced according to the method of claim 1, wherein said peptide has at least one amino acid that is labeled with deuterium wherein at least 10% of total occurrences of said amino acid in said peptide are labeled with deuterium.

16. A deuterated target peptide produced according to the method of claim 1, wherein said peptide has at least one amino acid that is labeled with deuterium wherein at least 90% of total occurrences of said amino acid in said peptide are labeled with deuterium.

17. A deuterated target peptide produced according to the method of claim 1, wherein said peptide has at least one amino acid that is labeled with deuterium wherein said labeled amino acid is located at a biologically active site within said peptide.

18. A deuterated target peptide according to claim 17, wherein said biologically active site within said peptide is selected from the group consisting of a binding site, an enzymatic active site, a substrate site for enzymatic activity, an allosteric site, or a biologically labile site.

19. A deuterated fusion peptide comprising an affinity tag, a cleavable tag, and a target peptide, wherein said deuterated fusion peptide contains at least one amino acid that is labeled with deuterium.

20. The deuterated fusion peptide of claim 19, wherein said cleavable tag is selected from the group consisting of is Trp, His-Met, Pro-Met, and an unnatural amino acid.

21. The deuterated fusion peptide of claim 19, wherein said peptide further comprises an inclusion-body directing tag.

22. A method of evaluating the commercial market for a deuterated target peptide comprising
a) producing a deuterated target peptide according to the method of claim 1;
b) making sample amounts of the deuterated target peptide available for no cost or minimal cost; and
c) measuring the number of requests for the deuterated target peptide over a period of time.

23. A composition comprising:
a) a cell containing a vector comprising
i) a nucleotide sequence encoding an affinity tag;
ii) a nucleotide sequence encoding a cleavable tag; and
iii) a nucleotide sequence encoding a target peptide;
wherein said nucleotides are arranged in operable combination and further wherein expression of the operable combination results in a fusion protein comprising an affinity tag, a cleavable tag, and a target peptide;
b) deuterium-containing culture medium.

24. The composition of claim 23, wherein said vector further comprises a nucleotide sequence encoding an inclusion-body directing tag.

25. The composition of claim 23, wherein said deuterium-containing culture medium contains at least one amino acid or amino acid precursor with at least one non-exchangable hydrogen replaced with deuterium.

26. A kit comprising the composition according to claim 23.
